(19) United States
(12) Patent Application Publication
(10) Pub. No.: US 2003/0167512 A1
(43) Pub. Date: Sep. 4, 2003

(54) Method of determining the presence of a trait in a plant by transfecting a nucleic acid sequence of a donor plant into a different host plant in a positive orientation

(76) Inventors: Monto H. Kumagai, Davis, CA (US); Guy R. Della-Cioppa, Vacaville, CA (US); Robert L. Erwin, Vacaville, CA (US); David R. McGee, Vacaville, CA (US)

Correspondence Address: Large Scale Biology Corporation, Suite 1000, 3333 Vaca Valley Parkway, Vacaville, CA, 95688, US

(21) Appl. No.: 10/236,508

(22) Filed: Sep. 6, 2002

Related U.S. Application Data
(63) Continuation of application No. 09/359,305, filed on Jul. 21, 1999.
Application No. 09/359,305 is a continuation-in-part of application No. 09/232,170, filed on Jan. 15, 1999, now abandoned.
Application No. 09/232,170 is a continuation-in-part of application No. 09/008,186, filed on Jan. 16, 1998.

(30) Foreign Application Priority Data
Jan. 15, 1999 (WO) PCT/US99/01164

(51) Int. Cl. A01H 1/00; C12N 15/87; C12N 5/04

(52) U.S. Cl. 800/278; 435/468; 435/419

(57) ABSTRACT

The present invention provides a method of compiling a plant positive sense functional gene profile, a method of changing the phenotype or biochemistry of a plant, a method of determining a change in phenotype or biochemistry of a plant, and a method of determining the presence of a trait in plant. The methods comprise expressing transiently a nucleic acid sequence of a plant into the cytoplasm of a host plant in a plus sense orientation to affect phenotypic or biochemical changes in the host plant. The nucleic acid sequence does not need to be isolated, identified or characterized prior to transfection into the host plant. A viral vector functional genomic screen has been developed to identify nucleotide sequences in transfected plants by systemically overproducing a new protein, or enhancing or suppressing the endogenous gene expression in a plus sense mechanism. Once the presence of a trait in a plant is identified by phenotypic or biochemical changes in the host plant, the nucleic acid insert in the cDNA clone or in the vector that results in the changes is then sequenced.

This application is a Continuation application of U.S. patent application Ser. No. 09/359,305, filed on Jul. 21, 1999, which is a Continuation-In-Part application of U.S. patent application Ser. No. 09/232,170, filed on Jan. 15, 1999, which is a Continuation-In-Part application of U.S. patent application Ser. No. 09/008,186, filed on Jan. 16, 1998. All the above applications are incorporated herein by reference.

### FIELD OF THE INVENTION

The present invention relates generally to the field of molecular biology and plant genetics. Specifically, the present invention relates to a method for determining the presence of a trait in a plant and a method of changing the phenotype or biochemistry of a plant, by expressing transiently a nucleic acid sequence of a donor plant in a positive orientation in a host plant. This invention is exemplified by a nucleic acid sequence comprising a GTP binding protein open reading frame having an positive orientation.

### BACKGROUND OF THE INVENTION

Great interest exists in launching genome projects in plants comparable to the human genome project. Valuable and basic agricultural plants, including by way of example but without limitation, corn, soybeans and rice are targets for such projects because the information obtained thereby may prove very beneficial for increasing world food production and improving the quality and value of agricultural products. The United States Congress is considering launching a corn genome project. By helping to unravel the genetics hidden in the corn genome, the project could aid in understanding and combating common diseases of grain crops. It could also provide a big boost for efforts to engineer plants to improve grain yields and resist drought, pests, salt, and other extreme environmental conditions. Such advances are critical for a world population expected to double by 2050. Currently, there are four species which provide 60% of all human food: wheat, rice, corn, and potatoes, and the strategies for increasing the productivity of these plants is dependent on rapid discovery of the presence of a trait in these plants, and the function of unknown gene sequences in these plants.

One strategy that has been proposed to assist in such efforts is to create a database of expressed sequence tags (ESTs) that can be used to identify expressed genes. Accumulation and analysis of expressed sequence tags (ESTs) have become an important component of genome research. EST data may be used to identify gene products and thereby accelerate gene cloning. Various sequence databases have been established in an effort to store and relate the tremendous amount of sequence information being generated by the ongoing sequencing efforts. Some have suggested sequencing 500,000 ESTs for corn and 100,000 ESTs each for rice, wheat, oats, barley, and sorghum. Efforts at sequencing the genomes of plant species will undoubtedly rely upon these computer databases to share the sequence data as it is generated. Arabidopsis thaliana may be an attractive target discovery of a trait and for gene function discovery because a very large set of ESTs have already been produced in this organism, and these sequences tag more than 50% of the expected Arabidopsis genes.

Potential use of the sequence information so generated is enormous if gene function can be determined. It may become possible to engineer commercial seeds for agricultural use to convey any number of desirable traits to food and fiber crops and thereby increase agricultural production and the world food supply. Research and development of commercial seeds has so far focused primarily on traditional plant breeding, however there has been increased interest in biotechnology as it relates to plant characteristics. Knowledge of the genomes involved and the function of genes contained therein for both monocotyledonous and dicotyledonous plants is essential to realize positive effects from such technology.

The impact of genomic research in seeds is potentially far reaching. For example, gene profiling in cotton can lead to an understanding of the types of genes being expressed primarily in fiber cells. The genes or promoters derived from these genes may be important in genetic engineering of cotton fiber for increased strength or for “built-in” fiber color. In plant breeding, gene profiling coupled to physiological trait analysis can lead to the identification of predictive markers that will be increasingly important in marker assisted breeding programs. Mining the DNA sequence of a particular crop for genes important for yield, quality, health, appearance, color, taste, etc., are applications of obvious importance for crop improvement.

Work has been conducted in the area of developing suitable vectors for expressing foreign DNA and RNA in plant hosts. Ahlquist, U.S. Pat. Nos. 4,885,248 and 5,173,410 describes preliminary work done in devising transfer vectors which might be useful in transferring foreign genetic material into a plant host for the purpose of expression therein. All patent references cited herein are hereby incorporated by reference. Additional aspects of hybrid RNA viruses and RNA transformation vectors are described by Ahlquist et al. in U.S. Pat. Nos. 5,466,788, 5,602,242, 5,627,060 and 5,500,360, all of which are incorporated herein by reference. Donson et al., U.S. Pat. Nos. 5,316,931, 5,589,367 and 5,866,785, incorporated herein by reference, demonstrate for the first time plant viral vectors suitable for the systemic expression of foreign genetic material in plants. Donson et al. describe plant viral vectors having heterologous subgenomic promoters for the systemic expression of foreign genes. Carrington et al., U.S. Pat. No. 5,491,076, describe particular polyvirus vectors also useful for expressing foreign genes in plants. The expression vectors described by Carrington et al. are characterized by utilizing the unique ability of viral polyprotein proteases to cleave heterologous proteins from viral polyproteins. These include Polyviruses such as Tobacco Etch Virus. Additional suitable vectors are described in U.S. Pat. No. 5,811,653 and U.S. patent application Ser. No. 08/324,003, both of which are incorporated herein by reference.

Construction of plant RNA viruses for the introduction and expression of non-viral foreign genes in plants has also been demonstrated by Brisson et al., Methods in Enzymology 118:659 (1986), Guzman et al., Communications in Molecular Biology: Viral Vectors, Cold Spring Harbor Laboratory, pp. 172-189 (1988), Dawson et al., Virology 172:285-292 (1989), Takamatsu et al., EMBO J. 6:307-311 (1987), French et al., Science 231:1294-1297 (1986), and Takamatsu et al., FEBS Letters 269:73-76 (1990). However, these viral vectors have not been shown capable of systemic spread in the plant and expression of the non-viral foreign genes in the majority of plant cells in the whole plant. Moreover, many of these viral vectors have not proven stable for the maintenance of non-viral foreign genes. However, the viral vectors described by Donson et al., in U.S. Pat. Nos. 5,316,931, 5,589,367, and 5,866,785, Turpen in U.S. Pat. Nos. 5,811,653, and 5,889,191 Carrington et al. in U.S. Pat. No. 5,491,076, and in co-pending U.S. patent application Ser. No. 08/324,003, have proven capable of infecting plant cells with foreign genetic material and systemically spreading in the plant and expressing the non-viral foreign genes contained therein in plant cells locally or systemically. All patents, patent applications, and references cited in the instant application are hereby incorporated by reference.

The expression of virus-derived sense or antisense RNA in transgenic plants provides an enhanced or reduced expression of an endogenous gene. In most cases, introduction and subsequent expression of a transgene will increase (with a sense RNA) or decrease (with an antisense RNA) the steady-state level of a specific gene product (Curr. Opin. Cell Biol., 7: 399-405, (1995)). There is also evidence that inhibition of endogenous genes occurs in transgenic plants containing sense RNA (Van der Krol et al., Plant Cell 2(4):291-299 (1990), Napoli et al., Plant Cell 2:279-289 (1990) and Fray et al., Plant Mol. Biol. 22:589-602 (1993)). The posttranscriptional gene silencing mechanism is typified by the highly specific degradation of both the transgene mRNA and the target RNA, which contains either the same or complementary nucleotide sequences. In cases that the silencing transgene is the same sense as the target endogenous gene or viral genomic RNA, it has been suggested that a plant-encoded RNA-dependent RNA polymerase makes a complementary strand from the transgene mRNA and that the small cRNAs potentiate the degradation of the target RNA. Antisense RNA and the hypothetical cRNAs have been proposed to act by hybridizing with the target RNA to either make the hybrid a substrate for double-stranded (ds) RNases or arrest the translation of the target RNA (Baulcombe, Plant Mol. Biol. 32: 79-88 (1996)). It is also proposed that this downregulation or “co-suppression” by the sense RNA might be due to the production of antisense RNA by readthrough transcription from distal promoters located on the opposite strand of the chromosomal DNA (Grierson et al., Trends Biotechnol. 9:122-123 (1993)).

Waterhouse et al (Proc. Natl. Acad. Sci. USA. 10: 13959-64 (1998)) prepared transgenic tobacco plants containing sense or antisense constructs. Pro[s] and Pro[a/s] constructs contained the PVY nuclear inclusion Pro ORF in the sense and antisense orientations, respectively. The Pro[s]-stop construct contained the PVY Pro ORF in the sense orientation but with a stop codon three codons downstream from the initiation codon. Waterhouse et al show when the genes of those constructs were transformed into plants, the plants exhibited immunity to the virus form which the transgene was dirived. Smith et al (Plant Cell, 6: 1441-1453, (1994)) prepared a tobacco transgenic plant containing the potato virus Y (PVY) coat protein (CP) open reading frame, which produced an mRNA rendered untranslatable by introduction of a stop codon immediately after the initiation codon. The expression of the untranslatable sense RNA inversely correlated with the virus resistance of the transgenic plant. Kumagai et al (Proc. Natl. Acad. Sci. USA 92:1679 (1995)) report that gene expression in transfected Nicotiana benthamiana was cytoplasmic inhibited by viral delivery of a RNA of a known sequence derived from cDNA encoding tomato (Lycopersicon esculentum) phytoene desaturase in a positive sense or an antisense orientation.

The plus sense and antisense technology can be used to develop a functional genomic screening of a plant of interest. The plus sense technology is applied in this invention to provide a method of discovering the presence of a trait in a plant and to determine the function and sequence of a nucleic acid of a plant by expressing the nucleic acid sequence that has not been identified in a different host plant. GTP-binding proteins exemplify this invention. In eukaryotic cells, GTP-binding proteins function in a variety of cellular processes, including signal transduction, cytoskeletal organization, and protein transport. Low molecular weight (20-25 K Daltons) of GTP-binding proteins include ras and its close relatives (for example, Ran), rho and its close close relatives, the rab family, and the ADP-ribosylation factor (ARF) family. The heterotrimeric and monomeric GTP-binding proteins that may be involved in secretion and intracellular transport are divided into two structural classes: the rab and the ARF families. Ran, a small soluble GTP-binding protein, has been shown to be essential for the nuclear translocation of proteins and it is also thought to be involved in regulating cell cycle progression in mammalian and yeast cells. The cDNAs encoding GTP binding proteins have been isolated from a variety of plants including rice, barley, corn, tobacco, and A. thaliana. For example, Verwoert et al. (Plant Molecular Biol. 27:629-633 (1995)) report the isolation of a Zea mays cDNA clone encoding a GTP-binding protein of the ARF family by direct genetic selection in an E. coli fabD mutant with a maize cDNA expression library. Regad et al. (FEBS 2:133-136 (1993)) isolated a cDNA clone encoding the ARF from a cDNA library of Arabidopsis thaliana cultured cells by randomly selecting and sequencing cDNA clones. Dallmann et al (Plant Molecular Biol. 19:847-857 (1992)) isolated two cDNAs encoding small GTP-binding proteins from leaf cDNA libraries using a PCR approach. Dallmann et al. prepared leaf cDNAs and use them as templates in PCR amplifications with degenerated oligonucleotides corresponding to the highly conserved motifs, found in members of the ras superfamily, as primers. Haizel et al., (Plant J., 11:93-103 (1997)) isolated cDNA and genomic clones encoding Ran-like small GTP binding proteins from Arabidopsis cDNA and genomic libraries using a full-length tobacco Nt Ran1 cDNA as a probe. The present invention provides advantages over the above methods in identifying nucleic acid sequence encoding GTP binding proteins in that it only sequences clones that have a function and does not randomly sequence clones. The nucleic acid inserts in clones that have a function are labeled and used as probes to isolate a cDNA hybridizing to them.

The present invention provides a method for discovering the presence of a trait in a plant by expressing a nucleic acid sequence in a positive orientation in a host plant. Once the presence of a trait is identified by phenotypic or biochemical changes, the nucleic acid insert in the cDNA clone or in the vector is then sequenced. The present method provides a rapid method for determining the presence of a trait and identifying a nucleic acid sequence and its function in a plant by screening a transfected host plant for its change of function.

### SUMMARY OF THE INVENTION

The present invention is directed to a method of changing the phenotype or biochemistry of a plant, a method of determining a change in phenotype or biochemistry in a plant, and a method of determining the presence of a trait in a plant. The method comprising the steps of expressing transiently an unidentified RNA or DNA sequence of a donor plant in a positive orientation in a host plant, identifying changes in the host plant, and correlating the expression with the phenotypic changes. Alternatively, the method comprises the steps of expressing transiently a nucleic acid sequence of a donor plant in a positive orientation in a host plant, identifying changes in the host plant, and correlating the expression with the phenotypic changes, wherein the donor plant and the host plant belong to different genus, family, order, class, subdivision, or division. The invention is also directed to a method of making a functional gene profile in a host plant by transiently expressing a nucleic acid sequence library in a host plant, determining the phenotypic or biochemical changes in the host plant, identifying a trait associated with the change, identifying the donor gene associated with the trait, and identifying the homologous host gene, if any. The present invention is also directed to a method of determining the function of a nucleic acid sequence, including a gene, in a donor plant, by transfecting the nucleic acid sequence into a host plant in a manner so as to affect phenotypic changes in the host plant.

In one embodiment, recombinant viral nucleic acids are prepared to include the nucleic acid insert of a donor. The recombinant viral nucleic acids infect a host plant and produce plus sense RNAs in the cytoplasm which result in reduced or enhanced expression of endogenous cellular genes in the host plant. Once the presence of a trait is identified by phenotypic or biochemical changes, the function of the nucleic acid is determined. The nucleic acid insert in a cDNA clone or in a vector is then sequenced. The nucleic acid sequence is determined by a standard sequence analysis. This invention is examplied by a nucleic acid sequence comprising a GTP binding protein open reading frame having a positive orientation.

The present invention is also directed to a method of increasing yield of a grain crop. The method comprises expressing a nucleic acid sequence of a donor plant in a positive orientation in the cytoplasm of a grain crop, wherein said expressing results in stunted growth and increased seed production of the grain crop. A preferred method comprises the steps of cloning the nucleic acid sequence into a plant viral vector and infecting the grain crop with a recombinant viral nucleic acid comprising said nucleic acid sequence.

One aspect of the invention is a method of identifying and determining a nucleic acid sequence in a donor plant, the expression of which in a transfected host plant results in phenotypic or biochemical changes in the host plant. The method introduces the nucleic acid into the host plant by way of a viral nucleic acid such as a plant viral nucleic acid suitable to produce expression of the nucleic acid in the transfected host. One embodiment applies the principle of post-transcription gene silencing of the endogenous host gene, using positive sense RNAs. Particularly, this silencing function is useful for silencing a multigene family frequently found in plants. Another embodiment utilizes the overexpression of a plus sense RNA that results in overproduction of a protein to cause phenotypic or biochemical changes in a host plant.

Another aspect of the invention is to discover genes having the same function in different plants. The method starts with building a cDNA library or a genomic DNA or RNA library of a first plant. Then, a recombinant viral nucleic acid comprising a nucleic acid insert derived from the library is prepared and is used to infect a different host plant. The infected host plant is inspected for phenotypic or biochemical changes. The recombinant viral nucleic acid that results in phenotypic or biochemical changes in the host plant is identified and the sequence of the nucleic acid insert is determined by a standard method. Such nucleic acid sequence in the first plant has substantial sequence homology as that in the host plant: the nucleic acid sequences are conserved between the two plants. This invention provides a rapid means for elucidating the function and sequence of nucleic acids of interest; such rapidly expanding information can be subsequently utilized in the field of genomics.

In one embodiment, a nucleic acid is introduced into a plant host wherein the plant host may be a monocotyledonous or dicotyledonous plant, plant tissue or plant cell. Preferably, the nucleic acid is introduced by way of a recombinant plant viral nucleic acid. Preferred recombinant plant viral nucleic acids useful in the present invention comprise a native plant viral subgenomic promoter, a plant viral coat protein coding sequence, and at least one non-native nucleic acid sequence. Some viral vectors used in accordance with the present invention may be encapsidated by the coat proteins encoded by the recombinant plant virus. Recombinant plant viral nucleic acids or recombinant plant viruses are used to infect a plant host. The recombinant plant viral nucleic acid is capable of replication in the host, localized or systemic spread in the host, and transcription or expression of the nonnative nucleic acid in the host to produce a phenotypic or biochemical change. Any suitable vector constructs useful to produce localized or systemic expression of nucleic acids in host plants are within the scope of the present invention.

### BRIEF DESCRIPTION OF THE FIGURES

FIG. 1 depicts the vector TT01/PSY+ (SEQ ID NOs: 9 and 10).

FIG. 2 depicts the vector TT01A/PDS+.

FIG. 3 depicts the vector TT01A/CaCCS+ (SEQ ID NOs: 49 and 50).

FIG. 4 depicts the plasmid KS+TVCVK #23.

FIG. 5 depicts the plasmid pBS 735.

FIG. 6 depicts the plasmid pBS 740.

FIG. 7 depicts the plasmid pBS 740 AT #2441 (ATCC No.: PTA-326).

FIG. 8 shows the nucleotide sequence of 740 AT #2441 (SEQ ID NOs: 29 and 30).

FIG. 9 shows the nucleotide sequence alignment of 740 AT #2441 (SEQ ID NOs: 31) and AF017991 (SEQ ID NO: 51), a A. thaliana salt stress inducible small GTP binding protein RAN1.

FIG. 10 shows the nucleotide sequence alignment of 740 AT #2441 (SEQ ID NO: 31) and L16787 (SEQ ID NO: 32), a N. tabacum small GTP-binding protein.

FIG. 11 shows the amino acid comparison of 740 AT #2441 to a tobacco RAN-B1 GTP binding protein (SEQ ID NOs: 33 and 34).

FIG. 12 shows the pBS 740 AT #1191 (SEQ ID NOs: 35 and 36) plasmid map.

FIG. 13 shows the nucleotide and amino acid sequence of 740 AT #1191.

FIG. 14 depicts the plasmid pBS 740 AT #855 (ATCC No.: PTA-332).

FIG. 15 shows the nucleotide sequence alignment of 740 AT #855 to A. thaliana HAT7 homeobox protein ORF (U09340) (SEQ ID NOs: 37, 38 and 39).

### DETAILED DESCRIPTION OF THE INVENTION

The present invention is directed to a method of changing the phenotype or biochemistry of a plant, a method of determining a change in phenotype or biochemistry in a plant, a method of determining the presence of a trait in a plant, and a method of determining the function of a nucleic acid sequence. The presence of a trait is identified either in the infected host plant or in an uninfected host plant. The methods comprise the steps of expressing transiently a nucleic acid sequence in a positive orientation in a host organism such as a plant, a plant tissue or a plant cell, identifying changes in the host organism and correlating the expression and the changes. The nucleic acid sequence, which is expressed in the host plant, does not need to be identified, isolated, or characterized prior to the expression. The donor plant and the host plant can belong to different genus, family, order, class, subdivision, or division.

In one preferred embodiment, the method comprising the steps of (a) preparing a library of cDNAs, genomic DNAs, or a pool of RNAs of a donor plant, (b) constructing recombinant viral nucleic acids comprising a nucleic acid insert derived from said library, (c) infecting each host plant with one of the recombinant viral nucleic acids, (d) growing said infected host plant, and (e) determining changes in said host plant.

The invention is directed to a method of compiling a plant positive sense functional gene profile. The method comprises (a) preparing a vector library of DNA or RNA sequences from a donor plant, each sequence being in a positive sense orientation; (b) infecting a plant host with a vector; (c) transiently expressing the donor plant DNA or RNA sequence in the growing plant host; (d) determining one or more phenotypic or biochemical changes in the plant host, if any; (e) identifying an associated trait where a phenotypic or biochemical change occurs; (f) identifying a donor plant gene associated with the trait; (g) identifying a plant host gene, if any, associated with the trait; and (h) repeating steps (b)-(g) until a positive sense functional gene profile of the plant host and/or of the donor plant is compiled.

The present method has the advantages that the nucleic acid sequence does not need to be identified or known prior to infecting a host plant with a recombinant viral nucleic acid comprising the nucleic acid sequence. Once changes in the host plant is observed, the nucleic acid sequence can be determined by further identifying the recombinant viral nucleic acid that results in changes in the host, and analyzing the sequence of the nucleic acid insert in the recombinant viral nucleic acid that results in changes in the host.

The present invention provides a method of infecting a host plant by a recombinant plant viral nucleic acid which contains one or more non-native nucleic acid sequences, or by a recombinant plant virus containing a recombinant plant viral nucleic acid. The non-native nucleic acids are subsequently transcribed or expressed in the infected host plant in a plus sense orientation, which results in (a) overexpressing a new protein, (b) inhibiting an endogeneous gene expression, or (c) enhancing an endogeneous gene expression, in the host plant. The inhibition of an endogeneous gene may result from co-suppression by the production of antisense RNA by readthrough transcription from distal promoters located on the opposite strand of the chromosomal DNA. The inhibition may also result from the expression of a partial cDNA gene, which sometimes lacks of a start codon or has a stop codon close to the start codon. The inhibition may also result from the expression of a nucleic acid sequence encoding a 3′- or 5′-untranslated region similar or identical to that of the endogeneous gene. The expression of the non-native nucleic acid sequences result in changing phenotypic traits in the host plant, affecting biochemical pathways within the plant, or affecting endogenous gene expression within the plant.

In one embodiment, a nucleic acid is introduced into a plant host by way of a recombinant viral nucleic acid. Such recombinant viral nucleic acids are stable for the maintenance and transcription or expression of non-native nucleic acid sequences and are capable of systemically transcribing or expressing such non-native sequences in the plant host. Preferred recombinant plant viral nucleic acids useful in the present invention comprise a native plant viral subgenomic promoter, a plant viral coat protein coding sequence, and at least one non-native nucleic acid sequence.

In a second embodiment, plant viral nucleic acid sequences are characterized by the deletion of a native coat protein coding sequence. The plant viral nucleic acid sequence comprises a non-native plant viral coat protein coding sequence and a non-native promoter, preferably the subgenomic promoter of the non-native coat protein coding sequence. Such plant viral nucleic acid sequence is capable of expressing in a plant host, packaging the recombinant plant viral nucleic acid, and ensuring a systemic infection of the host by the recombinant plant viral nucleic acid. The recombinant plant viral nucleic acid may contain one or more additional native or non-native subgenomic promoters. Each non-native subgenomic promoter is capable of transcribing or expressing adjacent genes or nucleic acid sequences in the plant host and incapable of recombination with each other and with native subgenomic promoters. One or more non-native nucleic acids may be inserted adjacent to the native plant viral subgenomic promoter or the native and non-native plant viral subgenomic promoters if more than one nucleic acid sequence is included. Moreover, two or more heterologous non-native subgenomic promoters may be used. The non-native nucleic acid sequences may be transcribed or expressed in the host plant under the control of the subgenomic promoter to produce the products of the nucleic acids of interest.

In a third embodiment, plant recombinant viral nucleic acids comprise a native coat protein coding sequence instead of a non-native coat protein coding sequence, placed adjacent one of the non-native coat protein subgenomic promoters.

In a fourth embodiment, plant recombinant viral nucleic acids comprise a native coat protein gene adjacent its native subgenomic promoter, one or more non-native subgenomic promoters, and at least one non-native nucleic acid sequence. The native plant viral subgenomic promoter initiates transcription of the plant viral coat protein sequence. The non-native subgenomic promoters are capable of transcribing or expressing adjacent genes in a plant host and are incapable of recombination with each other and with native subgenomic promoters. Non-native nucleic acid sequences may be inserted adjacent the non-native subgenomic plant viral promoters such that the sequences are transcribed or expressed in the host plant under control of the subgenomic promoters to produce a product of the non-native nucleic acid. Alternatively, the native coat protein coding sequence may be replaced by a non-native coat protein coding sequence.

The viral vectors used in accordance with the present invention may be encapsidated by the coat proteins encoded by the recombinant plant virus. The recombinant plant viral nucleic acid or recombinant plant virus is used to infect a host plant. The recombinant plant viral nucleic acid is capable of replication in the host, localized or systemic spread in the host, and transcription or expression of the non-native nucleic acid in the host to affect a phenotypic or biochemical change in the host.

In one embodiment, recombinant plant viruses are used which encode for the expression of a fusion between a plant viral coat protein and the amino acid product of the nucleic acid of interest. Such a recombinant plant virus provides for high level expression of a nucleic acid of interest. The location or locations where the viral coat protein is joined to the amino acid product of the nucleic acid of interest may be referred to as the fusion joint. A given product of such a construct may have one or more fusion joints. The fusion joint may be located at the carboxyl terminus of the viral coat protein or the fusion joint may be located at the amino terminus of the coat protein portion of the construct. In instances where the nucleic acid of interest is located internal with respect to the 5′ and 3′ residues of the nucleic acid sequence encoding for the viral coat protein, there are two fusion joints. That is, the nucleic acid of interest may be located 5′, 3′, upstream, downstream or within the coat protein. In some embodiments of such recombinant plant viruses, a “leaky” start or stop codon may occur at a fusion joint which sometimes does not result in translational termination. A more detailed description of some recombinant plant viruses according to this embodiment of the invention may be found in co-pending U.S. patent application Ser. No. 08/324,003 the disclosure of which is incorporated herein by reference.

The present invention is not intended to be limited to any particular viral constructs, but rather to include all operable constructs. Specifically, those skilled in the art may choose to transfer DNA or RNA of any size up to and including an entire genome in a plant into a host organism in order to determine the presence of a trait in the plant. Those skilled in the art will understand that the recited embodiments are representative only. All operable constructs useful to produce localized or systemic expression of nucleic acids in a plant are within the scope of the present invention.

The chimeric genes and vectors and recombinant plant viral nucleic acids used in this invention are constructed using techniques well known in the art. Suitable techniques have been described in Sambrook et al. (2nd ed.), Cold Spring Harbor Laboratory, Cold Spring Harbor (1982, 1989); Methods in Enzymol. (Vols. 68, 100, 101, 118, and 152-155) (1979, 1983, 1986 and 1987); and DNA Cloning, D. M. Clover, Ed., IRL Press, Oxford (1985). Medium compositions have been described by Miller, J., Experiments in Molecular Genetics, Cold Spring Harbor Laboratory, New York (1972), as well as the references previously identified, all of which are incorporated herein by reference. DNA manipulations and enzyme treatments are carried out in accordance with manufacturers' recommended procedures in making such constructs.

The first step in producing recombinant plant viral nucleic acids is to modify the nucleotide sequences of the plant viral nucleotide sequence by known conventional techniques such that one or more non-native subgenomic promoters are inserted into the plant viral nucleic acid without destroying the biological function of the plant viral nucleic acid. The subgenomic promoters are capable of transcribing or expressing adjacent nucleic acid sequences in a plant host infected by the recombination plant viral nucleic acid or recombinant plant virus. The native coat protein coding sequence may be deleted in some embodiments, placed under the control of a non-native subgenomic promoter in other embodiments, or retained in a further embodiment. If it is deleted or otherwise inactivated, a non-native coat protein gene is inserted under control of one of the non-native subgenomic promoters, or optionally under control of the native coat protein gene subgenomic promoter. The non-native coat protein is capable of encapsidating the recombinant plant viral nucleic acid to produce a recombinant plant virus. Thus, the recombinant plant viral nucleic acid contains a coat protein coding sequence, which may be native or a normative coat protein coding sequence, under control of one of the native or non-native subgenomic promoters. The coat protein is involved in the systemic infection of the plant host.

Viruses suitable for use according to the methods of the present invention include viruses from the tobamovirus group such as Tobacco Mosaic virus (TMV), Ribgrass Mosaic Virus (RGM), Cowpea Mosaic virus (CMV), Alfalfa Mosaic virus (AMV), Cucumber Green Mottle Mosaic virus watermelon strain (CGMMV-W) and Oat Mosaic virus (OMV) and viruses from the brome mosaic virus group such as Brome Mosaic virus (BMV), broad bean mottle virus and cowpea chlorotic mottle virus. Additional suitable viruses include Rice Necrosis virus (RNV), and geminiviruses such as Tomato Golden Mosaic virus (TGMV), Cassaya Latent virus (CLV) and Maize Streak virus (MSV). Each of these groups of suitable viruses is characterized below. However, the invention should not be construed as limited to using these particular viruses, but rather the present invention is contemplated to include all plant viruses at a minimum.

### Tobamovirus Group

The tobacco mosaic virus (TMV) is of particular interest to the instant invention because of its ability to express genes at high levels in plants. TMV is a member of the tobamovirus group. The TMV virion is a tubular filament, and comprises coat protein sub-units arranged in a single right-handed helix with the single-stranded RNA intercalated between the turns of the helix. TMV infects tobacco as well as other plants. TMV virions are 300 m×18 nm tubes with a 4 nm-diameter hollow canal, and consist of 2140 units of a single structural protein helically wound around a single RNA molecule. The genome is a 6395 base plus-sense RNA. The 5′-end is capped and the 3′-end contains a series of pseudoknots and a tRNA-like structure that will specifically accept histidine. The genomic RNA functions as mRNA for the production of proteins involved in viral replication: a 126-kDa protein that initiates 68 nucleotides from the 5′-terminus, and a 183-kDa protein synthesized by readthrough of an amber termination codon approximately 10% of the time. Only the 183-kDa and 126-kDa viral proteins are required for the TMV replication in trans. (Ogawa et al., Virology 185:580-584 (1991)). Additional proteins are translated from subgenomic size mRNA produced during replication (Dawson, Adv. Virus Res., 38:307-342 (1990)). The 30-kDa protein is required for cell-to-cell movement; the 17.5-kDa capsid protein is the single viral structural protein. The function of the predicted 54-kDa protein is unknown.

TMV assembly apparently occurs in plant cell cytoplasm, although it has been suggested that some TMV assembly may occur in chloroplasts since transcripts of ctDNA have been detected in purified TMV virions. Initiation of TMV assembly occurs by interaction between ring-shaped aggregates (“discs”) of coat protein (each disc consisting of two layers of 17 subunits) and a unique internal nucleation site in the RNA; a hairpin region about 900 nucleotides from the 3′-end in the common strain of TMV. Any RNA, including subgenomic RNAs containing this site, may be packaged into virions. The discs apparently assume a helical form on interaction with the RNA, and assembly (elongation) then proceeds in both directions (but much more rapidly in the 3′- to 5′-direction from the nucleation site).

Another member of the Tobamoviruses, the Cucumber Green Mottle Mosaic virus watermelon strain (CGMMV-W) is related to the cucumber virus. Nozu et al., Virology 45:577 (1971). The coat protein of CGMMV-W interacts with RNA of both TMV and CGMMV to assemble viral particles in vitro. Kurisu et al., Virology 70:214 (1976).

Several strains of the tobamovirus group are divided into two subgroups, on the basis of the location of the assembly of origin. Subgroup I, which includes the vulgare, OM, and tomato strain, has an origin of assembly about 800-1000 nucleotides from the 3′-end of the RNA genome, and outside the coat protein cistron. Lebeurier et al, Proc. Natl. Acad. Sci. USA 74:149 (1977); and Fukuda et al., Virology 101:493 (1980). Subgroup II, which includes CGMMV-W and cornpea strain (Cc) has an origin of assembly about 300-500 nucleotides from the 3′-end of the RNA genome and within the coat-protein cistron. The coat protein cistron of CGMMV-W is located at nucleotides 176-661 from the 3′-end. The 3′ noncoding region is 175 nucleotides long. The origin of assembly is positioned within the coat protein cistron. Meshi et al., Virology 27:54 (1983).

### Brome Mosaic Virus Group

Brome Mosaic virus (BMV) is a member of a group of tripartite, single-stranded, RNA-containing plant viruses commonly referred to as the bromoviruses. Each member of the bromoviruses infects a narrow range of plants. Mechanical transmission of bromoviruses occurs readily, and some members are transmitted by beetles. In addition to BV, other bromoviruses include broad bean mottle virus and cowpea chlorotic mottle virus.

Typically, a bromovirus virion is icosahedral, with a diameter of about 26 μm, containing a single species of coat protein. The bromovirus genome has three molecules of linear, positive-sense, single-stranded RNA, and the coat protein mRNA is also encapsidated. The RNAs each have a capped 5′-end, and a tRNA-like structure (which accepts tyrosine) at the 3′-end. Virus assembly occurs in the cytoplasm. The complete nucleotide sequence of BMV has been identified and characterized as described by Ahlquist et al., J. Mol. Biol. 153:23 (1981).

### Rice Necrosis Virus

Rice Necrosis virus is a member of the Potato Virus Y Group or Polyviruses. The Rice Necrosis virion is a flexuous filament comprising one type of coat protein (molecular weight about 32,000 to about 36,000) and one molecule of linear positive-sense single-stranded RNA. The Rice Necrosis virus is transmitted by Polymyxa oraminis (a eukaryotic intracellular parasite found in plants, algae and fungi).

### Geminiviruses

Geminiviruses are a group of small, single-stranded DNA-containing plant viruses with virions of unique morphology. Each virion consists of a pair of isometric particles (incomplete icosahedral), composed of a single type of protein (with a molecular weight of about 2.7-3.4×10⁴). Each geminivirus virion contains one molecule of circular, positive-sense, single-stranded DNA. In some geminiviruses (i.e., Cassaya latent virus and bean golden mosaic virus) the genome appears to be bipartite, containing two single-stranded DNA molecules.

### Potyviruses

Polyviruses are a group of plant viruses which produce polyprotein. A particularly preferred polyvirus is tobacco etch virus (TEV). TEV is a well characterized polyvirus and contains a positive-strand RNA genome of 9.5 kilobases encoding for a single, large polyprotein that is processed by three virus-specific proteinases. The nuclear inclusion protein “a” proteinase is involved in the maturation of several replication-associated proteins and capsid protein. The helper component-proteinase (HC-Pro) and 35-kDa proteinase both catalyze cleavage only at their respective C-termini. The proteolytic domain in each of these proteins is located near the C-terminus. The 35-kDa proteinase and HC-Pro derive from the N-terminal region of the TEV polyprotein.

The nucleic acid of any suitable plant virus can be utilized to prepare a recombinant plant viral nucleic acid for use in the present invention, and the foregoing are only exemplary of such suitable plant viruses. The nucleotide sequence of the plant virus can be modified, using conventional techniques, by insertion of one or more subgenomic promoters into the plant viral nucleic acid. The subgenomic promoters are capable of functioning in a specific host plant. For example, if the host is a tobacco plant, TMV, TEV, or other viruses containing suitable subgenomic promoter may be utilized. The inserted subgenomic promoters should be compatible with the viral nucleic acid and capable of directing transcription or expression of adjacent nucleic acid sequences in tobacco.

The native or non-native coat protein gene is included in the recombinant plant viral nucleic acid. When non-native nucleic acid is utilized, it may be positioned adjacent its natural subgenomic promoter or adjacent one of the other available subgenomic promoters. The non-native coat protein, as is the case for the native coat protein, is capable of encapsidating the recombinant plant viral nucleic acid and providing for systemic spread of the recombinant plant viral nucleic acid in a host plant. The coat protein is selected to provide a systemic infection in the plant host of interest. For example, the TMV-O coat protein provides systemic infection in N. benthamiana, whereas TMV-U1 coat protein provides systemic infection in N. tabacum.

The recombinant plant viral nucleic acid is prepared by cloning a viral nucleic acid. If the viral nucleic acid is DNA, it can be cloned directly into a suitable vector using conventional techniques. One technique is to attach an origin of replication to the viral DNA which is compatible with the cell to be transfected. If the viral nucleic acid is RNA, a full-length DNA copy of the viral genome is first prepared by well-known procedures. For example, the viral RNA is transcribed into DNA using reverse transcriptase to produce subgenomic DNA pieces, and a double-stranded DNA made using DNA polymerases. The cDNA is then cloned into appropriate vectors and cloned into a cell to be transfected. Alternatively, the cDNA is ligated into the vector and is directly transcribed into infectious RNA in vitro, the infectious RNA is then inoculated onto the plant host. The cDNA pieces are mapped and combined in a proper sequence to produce a full-length DNA copy of the viral RNA genome, if necessary. DNA sequences for the subgenomic promoters, with or without a coat protein gene, are then inserted into the nucleic acid at non-essential sites, according to the particular embodiment of the invention utilized. Non-essential sites are those that do not affect the biological properties of the plant viral nucleic acids. Since the RNA genome is the infective agent, the cDNA is positioned adjacent a suitable promoter so that the RNA is produced in the production cell. The RNA can be capped by the addition of a nucleotide in a 5′-5′ linkage using conventional techniques. (Dawson et. al., Proc. Natl. Acad. Sci. USA, 83: 1832 (1986)). One or more nucleotides may be added between the transcription start site of the promoter and the start of the cDNA of a viral nucleic acid to construct an infectious viral vector. In a preferred embodiment of the present invention, the inserted nucleotide sequence contains a G at the 5′-end. In one embodiment, the inserted nucleotide sequence is GNN, GTN, or their multiples, (GNN)ₓ or (GTN)ₓ. In addition, the capped RNA can be packaged in vitro with added coat protein from TMV to make assembled virions. These assembled virions can then be used to inoculate plants or plant tissues.

Alternatively, an uncapped RNA may also be employed in the embodiments of the present invention. Contrary to the practiced art in scientific literature and in an issued patent (Ahlquist et al., U.S. Pat. No. 5,466,788), uncapped transcripts for virus expression vectors are infective on both plants and in plant cells. Capping is not a prerequisite for establishing an infection of a virus expression vector in plants, although capping increases the efficiency of infection.

One feature of the recombinant plant viral nucleic acids useful in the present invention is that they further comprise one or more non-native nucleic acid sequences capable of being transcribed in a host plant. These nucleic acid sequences may be native nucleic acid sequences that occur in a host plant. Preferably, these nucleic acid sequences are non-native nucleic acid sequences that do not normally occur in a host plant. These nucleic acid sequences are derived from a donor plant, which is preferably genetically different from the host plant. The donor plant and the host plant may be genetically remote or unrelated: they may belong to different genus, family, order, class, subdivision, or division. Donor plants and host plants include plants of commercial interest, such as food crops, seed crops, oil crops, ornamental crops and forestry crops. For example, wheat, rice, corn, potatoes, barley, tobaccos, soybean canola, maize, oilseed rape, Arabidopsis, Nicotiana can be selected as a donor plant or a host plant. Host plants include those capable of being infected by an infectious RNA or a virus containing a recombinant viral nucleic acid. Preferred host plants include Nicotiana, preferably, Nicotiana benthamiana, or Nicotiana cleavlandii.

Transient Expression: Expression of a nucleic acid sequence in a host without insertion of the nucleic acid sequence into the host genome, such as by way of a viral vector.

To prepare a DNA insert comprising a nucleic acid sequence of a donor plant, the first step is to construct a library of cDNAs, genomic DNAs, or a pool of RNAs of a plant. Full-length cDNAs can be obtained from public or private repositories, for example, cDNA library of Arabidopsis thaliana can be obtained from the Arabidopsis Biological Resource Center. Alternatively, cDNA library can be prepared from a field sample by methods known to a person of ordinary skill, for example, isolating mRNAs and transcribing mRNAs into cDNAs by reverse transcriptase (see, e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987)). Genomic DNAs represented in BAC (bacterial artificial chromosome), YAC (yeast artificial chromosome), or TAC (transformation-competent artificial chromosome, Liu et. al., Proc. Natl. Acad. Sci. USA 96:6535-6540 (1999)) libraries can be obtained from public or private repositories, for example, the Arabidopsis Biological Resource Center. The BAC/YAC/TAC DNAs or cDNAs can be mechanically size-fractionated or digested by an enzyme to smaller fragments. The fragments are ligated to adapters with cohesive ends, and shotgun-cloned into recombinant viral nucleic acid vectors. Alternatively, the fragments can be blunt-end ligated into recombinant viral nucleic acid vectors. Recombinant plant viral nucleic acids containing a nucleic acid sequence derived from the cDNA library or genomic DNA library is then constructed using conventional techniques. The recombinant viral nucleic acid vectors produced comprise the nucleic acid insert derived from the donor plant. The nucleic acid sequence of the recombinant viral nucleic acid is transcribed as RNA in a host plant; the RNA is capable of regulating the expression of a phenotypic trait by a plus sense mechanism. Alternatively, the nucleic acid sequence in the recombinant plant viral nucleic acid may be transcribed and translated in the plant host to change a phenotypic trait. The nucleic acid sequence may also code for the expression of more than one phenotypic trait. Sequences from wheat, rice, corn, potato, barley, tobacco, soybean, canola, maize, oilseed rape, Arabidopsis, and other crop species may be selected as donor plants to assemble the DNA libraries. This method may thus be used to search for useful dominant gene phenotypes from DNA libraries through the gene expression.

A further alternative when creating the recombinant plant viral nucleic acid is to prepare more than one nucleic acid (i.e., to prepare the nucleic acids necessary for a multipartite viral vector construct). In this case, each nucleic acid would require its own origin of assembly. Each nucleic acid could be prepared to contain a subgenomic promoter and a non-native nucleic acid.

A host can be infected with a recombinant viral nucleic acid or a recombinant plant virus by conventional techniques. Suitable techniques include, but are not limited to, leaf abrasion, abrasion in solution, high velocity water spray, and other injury of a host as well as imbibing host seeds with water containing the recombinant viral RNA or recombinant plant virus. More specifically, suitable techniques include:

(a) Hand Inoculations. Hand inoculations are performed using a neutral pH, low molarity phosphate buffer, with the addition of celite or carborundum (usually about 1%). One to four drops of the preparation is put onto the upper surface of a leaf and gently rubbed.

(b) Mechanized Inoculations of Plant Beds. Plant bed inoculations are performed by spraying (gas-propelled) the vector solution into a tractor-driven mower while cutting the leaves. Alternatively, the plant bed is mowed and the vector solution sprayed immediately onto the cut leaves.

(c) Vacuum Infiltration. Inoculations may be accomplished by subjecting a host organism to a substantially vacuum pressure environment in order to facilitate infection.

(d) High Speed Robotics Inoculation. Especially applicable when the organism is a plant, individual organisms may be grown in mass array such as in microtiter plates. Machinery such as robotics may then be used to transfer the nucleic acid of interest.

(e) High Pressure Spray of Single Leaves. Single plant inoculations can also be performed by spraying the leaves with a narrow, directed spray (50 psi, 6-12 inches from the leaf) containing approximately 1% carborundum in the buffered vector solution.

(f) Ballistics (High Pressure Gun) Inoculation. Single plant inoculations can also be performed by particle bombardment. A ballistics particle delivery system (BioRad Laboratories, Hercules, (A) can be used to transfect plants such as N. benthamiana as described previously (Nagar et al., Plant Cell, 7:705-719 (1995)).

An alternative method for introducing a recombinant plant viral nucleic acid into a plant host is a technique known as agroinfection or Agrobacterium-mediated transformation (sometimes called Agro-infection) as described by Grimsley et al., Nature 325:177 (1987). This technique makes use of a common feature of Agrobacterium which colonizes plants by transferring a portion of their DNA (the T-DNA) into a host cell, where it becomes integrated into nuclear DNA. The T-DNA is defined by border sequences which are 25 base pairs long, and any DNA between these border sequences is transferred to the plant cells as well. The insertion of a recombinant plant viral nucleic acid between the T-DNA border sequences results in transfer of the recombinant plant viral nucleic acid to the plant cells, where the recombinant plant viral nucleic acid is replicated, and then spreads systemically through the plant. Agro-infection has been accomplished with potato spindle tuber viroid (PSTV) (Gardner et al., Plant Mol. Biol. 6:221 (1986); CaV (Grimsley et al., Proc. Natl. Acad. Sci. USA 83:3282 (1986)); MSV (Grimsley et al., Nature 325:177 (1987)), and Lazarowitz, S., Nucl. Acids Res. 16:229 (1988)) digitaria streak virus (Donson et al., Virology 162:248 (1988)), wheat dwarf virus (Hayes et al., J. Gen. Virol. 69:891 (1988)) and tomato golden mosaic virus (TGMV) (Elmer et al., Plant Mol. Biol. 10:225 (1988) and Gardiner et al., EMBO J. 7:899 (1988)). Therefore, agro-infection of a susceptible plant could be accomplished with a virion containing a recombinant plant viral nucleic acid based on the nucleotide sequence of any of the above viruses. Particle bombardment or electrosporation or any other methods known in the art may also be used.

Infection may also be attained by placing a selected nucleic acid sequence into an organism such as E. coli, or yeast, either integrated into the genome of such organism or not, and then applying the organism to the surface of the host organism. Such a mechanism may thereby produce secondary transfer of the selected nucleic acid sequence into a host organism. This is a particularly practical embodiment when the host organism is a plant. Likewise, infection may be attained by first packaging a selected nucleic acid sequence in a pseudovirus. Such a method is described in WO 94/10329, the teachings of which are incorporated herein by reference. Though the teachings of this reference may be specific for bacteria, those of skill in the art will readily appreciate that the same procedures could easily be adapted to other organisms.

After a host is infected with a recombinant viral nucleic acid comprising a nucleic acid insert derived from a library of cDNAs, genomic DNAs, or a pool of RNAs, one or more biochemical or phenotypic changes in a host plant is determined. The biochemical or phenotypic changes in the infected host plant is correlated to the biochemistry or phenotype of a host plant that is uninfected. Optionally, the biochemical or phenotypic changes in the infected host plant is further correlated to a host plant that is infected with a viral vector that contains a control nucleic acid of a known sequence in a positive orientation; the control nucleic acid has similar size but is different in sequence from the nucleic acid insert derived from the library. For example, if the nucleic acid insert derived from the library is identified as encoding a GTP binding protein in a plus sense orientation, a nucleic acid derived from a gene encoding green fluorescent protein can be used as a control nucleic acid. Green fluorescent protein is known not be have the same effect as the GTP binding protein when expressed in plants.

Those of skill in the art will readily understand that there are many methods to determine phenotypic or biochemical change in a plant and to determine the function of a nucleic acid, once the nucleic acid is localized or systemic expressed in a host plant. In a preferred embodiment, the phenotypic or biochemical trait may be determined by observing phenotypic changes in a host by methods including visual, morphological, macroscopic or microscopic analysis. For example, growth change such as stunting, hyperbranching, and necrosis; structure change such as vein banding, ring spot, etching; color change such as bleaching, chlorosis, or other color; and other changes such as marginal, mottled, patterning, punctate, and reticulate are easily detected. In another embodiment, the phenotypic or biochemical trait may be determined by complementation analysis, that is, by observing the endogenous gene or genes whose function is replaced or augmented by introducing the nucleic acid of interest. A discussion of such phenomenon is provided by Napoli et al., The Plant Cell 2:279-289 (1990). In a third embodiment, the phenotypic or biochemical trait may be determined by analyzing the biochemical alterations in the accumulation of substrates or products from enzymatic reactions according to any means known by those skilled in the art. In a fourth embodiment, the phenotypic or biochemical trait may be determined by observing any changes in biochemical pathways which may be modified in a host organism as a result of expression of the nucleic acid. In a fifth embodiment, the phenotypic or biochemical trait may be determined utilizing techniques known by those skilled in the art to observe inhibition of endogenous gene expression in the cytoplasm of cells as a result of expression of the nucleic acid. In a sixth embodiment, the phenotypic or biochemical trait may be determined utilizing techniques known by those skilled in the art to observe changes in the RNA or protein profile as a result of expression of the nucleic acid. In a seventh embodiment, the phenotypic or biochemical trait may be determined by selection of organisms such as plants capable of growing or maintaining viability in the presence of noxious or toxic substances, such as, for example herbicides and pharmaceutical ingredients.

Phenotypic traits in plant cells, which may be observed microscopically, macroscopically or by other methods, include improved tolerance to herbicides, improved tolerance to extremes of heat or cold, drought, salinity or osmotic stress; improved resistance to pests (insects, nematodes or arachnids) or diseases (fungal, bacterial or viral), production of enzymes or secondary metabolites; male or female sterility; dwarfness; early maturity; improved yield, vigor, heterosis, nutritional qualities, flavor or processing properties, and the like. Other examples include the production of important proteins or other products for commercial use, such as lipase, melanin, pigments, alkaloids, antibodies, hormones, pharmaceuticals, antibiotics and the like. Another useful phenotypic trait is the production of degradative or inhibitory enzymes, for example, enzymes preventing or inhibiting the root development in malting barley, or enzymes determining response or non-response to a systemically administered drug in a human. The phenotypic trait may also be a secondary metabolite whose production is desired in a bioreactor.

Biochemical changes can also be determined by analytical methods, for example, in a high-throughput, fully automated fashion using robotics. Suitable biochemical analysis may include MALDI-TOF, LC/MS, GC/MS, two-dimensional IEF/SDS-PAGE, ELISA or other methods of analyses. The clones in the plant viral vector library may then be functionally classified based on metabolic pathway affected or visual/selectable phenotype produced in the plant. This process enables the rapid determination of gene function for unknown nucleic acid sequences of a plant origin. Furthermore, this process can be used to rapidly confirm function of full-length DNA's of unknown gene function. Functional identification of unknown nucleic acid sequences in a plant library may then rapidly lead to identification of similar unknown sequences in expression libraries for other crop species based on sequence homology.

One useful means to determine the function of nucleic acids transfected into a host is to observe the effects of gene silencing. Traditionally, functional gene knockout has been achieved following inactivation due to insertion of transposable elements or random integration of T-DNA into the chromosome, followed by characterization of conditional, homozygous-recessive mutants obtained upon backcrossing. Some teachings in these regards are provided by WO 97/42210 which is herein incorporated by reference. As an alternative to traditional knockout analysis, an EST/DNA library from an organism, for example Arabidopsis thaliana, may be assembled into a plant viral transcription plasmid. The nucleic acid sequences in the transcription plasmid library may then be introduced into plant cells as part of a functional RNA virus which post-transcriptionally silences the homologous target gene. The EST/DNA sequences may be introduced into a plant viral vector in either the plus or minus sense orientation, and the orientation can be either directed or random based on the cloning strategy. A high-throughput, automated cloning scheme based on robotics may be used to assemble and characterize the library. In addition, double stranded RNA may also be an effective stimulator of gene silencing in transgenic plant. Gene silencing of plant genes may be induced by delivering an RNA capable of base pairing with itself to form double stranded regions. This approach could be used with any plant gene to assist in the identification of the function of a particular gene sequence.

The present invention provides a method to produce transfected plants containing viral-derived plus sense RNA in the cytoplasm. Such method is much faster than the time required to obtain genetically engineered transgenic plants. Systemic infection and expression of viral plus sense RNA in the cytoplasm occurs as short as four days post inoculation, whereas it takes several months or longer to create a single transgenic plant. The invention provides a method to identify genes involved in the regulation of plant growth by inhibiting or enhancing the expression of specific endogenous genes using viral vectors, which replicate solely in the cytoplasm. This invention provides a method to characterize specific genes and biochemical pathways in donor plants or in host plants using an RNA viral vector.

The invention is also directed to a method of determining a nucleic acid sequence in a donor plant, which has the same function as that in a host plant, by transfecting a nucleic acid sequence derived from a donor plant into a plant host. In one preferred embodiment, the method comprising the steps of (a) preparing a library of cDNAs, genomic DNAs, or a pool of RNAs of the donor plant, (b) constructing recombinant viral nucleic acids comprising a nucleic acid insert derived from the library, (c) infecting each host plant with one of the recombinant viral nucleic acids, (d) growing the infected host plant, (e) determining one or more changes in the host plant, (f) identifying the recombinant viral nucleic acid that results in changes in the host, (g) determining the sequence of the nucleic acid insert in the recombinant viral nucleic acid that results in changes in the host, and (h) determining the sequence of an entire open reading frame of the donor from which the nucleic acid insert is derived.

The invention is further directed to a method of determining a nucleic acid sequence in a host plant, which has the same function as that in a donor plant, by transfecting a nucleic acid sequence derived from a donor plant into a host plant. In one preferred embodiment, the method comprising the steps of (a) preparing a library of cDNAs, genomic DNAs, or a pool of RNAs of the donor plant, (b) constructing recombinant viral nucleic acids comprising a nucleic acid insert derived from the library, (c) infecting each host plant with one of said recombinant viral nucleic acids, (d) growing the infected host plant, (e) determining one or more changes in the host plant, (f) identifying the recombinant viral nucleic acid that results in changes in the host, (g) determining the sequence of the nucleic acid insert in the recombinant viral nucleic acid that results in changes in the host, and (h) determining the sequence of an entire open reading frame of a gene in the host plant, the expression of which is affected by the insert. The sequence of the nucleic acid insert in the cDNA clone or in the viral vector can be determined by a standard method, for example, by dideoxy termination using double stranded templates (Sanger et al., Proc., Natl. Acad. Sci. USA 74:5463-5467 (1977)). Once the sequence of the nucleic acid insert is obtained, the sequence of an entire open reading frame of a gene can be determined by probing filters containing full-length cDNAs from the cDNA library with the nucleic acid insert labeled with radioactive, fluorescent or enzyme molecules. The sequence of an entire open reading frame of a gene can also be determined by RT-PCR (Methods Mol. Biol., 89: 333-358 (1998))

One problem of changing the expression of a gene in a plant host is that many plant genes exist in multigene families. Therefore, effective changing a gene function may be especially problematic. According to the present invention, however, nucleic acids may be inserted into the viral genome to effectively change a particular gene function or to change the function of a multigene family.

A detailed discussion of some aspects of the “gene silencing” effect is provided in the co-pending patent application, U.S. patent application Ser. No. 08/260,546 (WO95/34668 published 12/21195) the disclosure of which is incorporated herein by reference. RNA can reduce the expression of a target gene through inhibitory RNA interactions with target mRNA that occur in the cytoplasm and/or the nucleus of a cell.

An EST/cDNA library from a plant such as Arabidopsis thaliana may be assembled into a plant viral transcription plasmid background. The cDNA sequences in the transcription plasmid library can then be introduced into plant cells as cytoplasmic RNA in order to post-transcriptionally silence the endogenous genes. The EST/cDNA sequences may be introduced into the plant viral transcription plasmid in either the plus or anti-sense orientation (or both), and the orientation can be either directed or random based on the cloning strategy. A high-throughput, automated cloning strategy using robotics can be used to assemble the library. The EST clones can be inserted behind a duplicated subgenomic promoter such that they are represented as subgenomic transcripts during viral replication in plant cells. Alternatively, the EST/cDNA sequences can be inserted into the genomic RNA of a plant viral vector such that they are represented as genomic RNA during the viral replication in plant cells. The library of EST clones is then transcribed into infectious RNAs and inoculated onto a host plant susceptible to viral infection. The viral RNAs containing the EST/cDNA sequences contributed from the original library are now present in a sufficiently high concentration in the cytoplasm of host plant cells such that they cause posttranscriptional gene silencing of the endogenous gene in a host plant. Since the replication mechanism of the virus produces both sense and antisense RNA sequences, the orientation of the EST/cDNA insert is normally irrelevant in terms of producing the desired phenotype in a host plant.

It is known that silencing of endogenous genes can be achieved with homologous sequences from the same family. For example, Kumagai et al., (Proc. Natl. Acad. Sci. USA 92:1679 (1995)) report that the Nicotiana benthamiana gene for phytoene desaturase (PDS) was silenced by transfection with a viral RNA derived from a clone containing a partial tomato (Lycopersicon esculentum) cDNA encoding PDS being in an antisense or in a positive sense orientation. This paper is incorporated here by reference. Kumagai et al. demonstrate that gene encoding partial PDS from one plant can be silenced by transfection a host plant with a nucleic acid of a known sequence, namely, a PDS gene, from a donor plant of the same family. The present invention provides a method of enhancing or reducing the expression of an endogeneous gene in a host plant, or overexpressing a new protein in a host plant, by transfecting the host plant with a viral nucleic acid comprising a nucleic acid insert derived from a cDNA library, or a genomic DNA or RNA library. Different from Kumagai et al., the sequence of the nucleic acid insert in the present invention is not identified prior to the transfection. Another feature of the present invention is that it provides a method to change the expression of a gene of a different family; the plus sense transcript of one plant results in enhancing or reducing expression of the endogenous gene or multigene family of a plant of a different genus, family, order, class, subdivision, or division. The present invention is exemplified by overproduction of a GTP binding proteins. The present invention demonstrates that genes of one plant, such as Arabidopsis, which encode a GTP binding protein Ran, can be overexpressed in a different host plant by transfection the host plant with infectious RNAs containing cDNA inserts from Arabidopsis cDNA library in a plus orientation, and result in host plant stunting.

The present invention also provides a method of isolating a conserved gene such as a gene encoding a GTP binding protein, from rice, barley, corn, soybean, maize, oilseed, and other plant of commercial interest, using another gene having homology with gene being isolated. Libraries containing full-length cDNAs from a donor plant such as rice, barley, corn, soybean and other important crops can be obtained from public and private sources or can be prepared from plant mRNAs. The cDNAs are inserted in viral vectors or in small subcloning vectors such as pBluescript (Strategene), pUC18, M13, or pBR322. Transformed bacteria are then plated and individual clones selected by a standard method. The bacteria transformants or DNAs are rearrayed at high density onto membrane filters or glass slides. Full-length cDNAs encoding GTP binding proteins can be identified by probing filters or slides with labeled nucleic acid inserts that result in changes in a host plant, or with labeled probes prepared from DNAs encoding GTP binding proteins from Arabidopsis. Useful labels include radioactive, fluorescent, or chemiluminescent molecules, enzymes, etc.

Alternatively, genomic libraries containing sequences from rice, barley, corn, soybean and other important crops can be obtained from public and private sources, or be prepared from plant genomic DNAs. BAC clones containing entire plant genomes have been constructed and organized in a minimal overlapping order. Individual BACs are sheared to fragments and directly cloned into viral vectors. Clones that completely cover

an entire BAC form a BAC viral vector sublibrary. Genomic clones can be identified by probing filters containing BACs with labeled nucleic acid inserts that result in changes in a host plant, or with labeled probes prepared from DNAs encoding GTP binding proteins from Arabidopsis. Useful labels include radioactive, fluorescent, or chemiluminescent molecules, enzymes, etc. BACs that hybridize to the probe are selected and their corresponding BAC viral vectors are used to produce infectious RNAs. Plants that are transfected with the BAC sublibrary are screened for change of function, for example, change of growth rate or change of color. Once the change of function is observed, the inserts from these clones or their corresponding plasmid DNAs are characterized by dideoxy sequencing. This provides a rapid method to obtain the genomic sequence for a plant protein, for example, a GTP binding protein. Using this method, once the DNA sequence in one plant such as Arabidopsis thaliana is identified, it can be used to identify conserved sequences of similar function that exist in other plant libraries.

In some embodiments of the instant invention, methods to increase the representation of gene sequences in virus expression libraries may also be achieved by bypassing the genetic bottleneck of propagation in bacterial cells. For example, cell-free methods may be used to assemble sequence libraries or individual arrayed sequences into virus expression vectors and reconstruct an infectious virus, such that the final ligation product can be transcribed and the resulting RNA can be used for plant, plant tissue or plant cell inoculation/infection. A more detailed discussion is presented in a co-pending and co-owned U.S. patent application Ser. No. 09/359,303 (Padgett et al., Attorney Docket No. 08010137US03, filed Jul. 21, 1999), the entire disclosure of which is incorporated herein by reference.

Large amounts of DNA sequence information are being generated in the public domain and may be entered into a relational database. Links may be made between sequences from various species predicted to carry out similar biochemical or regulatory functions. Links may also be generated between predicted enzymatic activities and visually displayed biochemical and regulatory pathways. Likewise, links may be generated between predicted enzymatic or regulatory activity and known small molecule inhibitors, activators, substrates or substrate analogs. Phenotypic data from expression libraries expressed in transfected hosts may be automatically linked within such a relational database. Genes with similar predicted roles of interest in other crop plants may be rapidly discovered.

A complete classification scheme of gene functionality for a fully sequenced eukaryotic organism has been established for yeast. This classification scheme may be modified for plants and divided into the appropriate categories. Such organizational structure may be utilized to rapidly identify herbicide target loci which may confer dominant lethal phenotypes, and thereby is useful in helping to design rational herbicide programs.

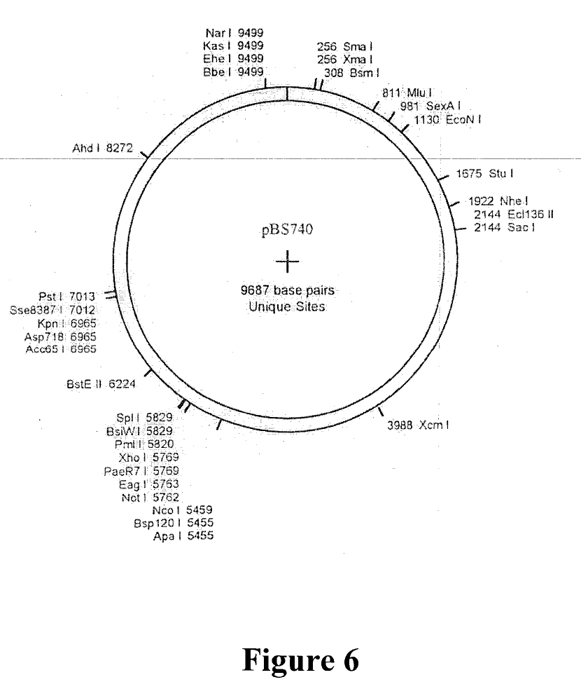

This invention is examplied by setting up a functional genomics screen using a Tobacco Mosaic Virus having a TMV-O coat protein capsid for infection of Nicotiana benthamiana, a plant related to the common tobacco plant. An Arabidopsis thaliana cDNA library is obtained from the Arabidopsis Biological Resource Center. Blue Script® phagemid vectors are recovered by not1 digestion. cDNA is transformed into c plasmid pBS740 which transforms competent E. Coli cells. cDNA of 500 to 1000 base pairs is inserted in the plus sense orientation. As in FIG. 6 until all of the cDNA from the cDNA library is represented on viral vectors. Each viral vector is sprayed onto the leaf of a two-week old N. benthamiana plant host with sufficient force to cause tissue injury and localized viral infection. Each infected plant is grown side by side with an uninfected plant and a plant infected with a null insert vector as controls. All plants are grown in an artificial environment having 16 hours of light and 8 hours of dark. Lumens are approximately equal on each plant. At intervals of 2 days, a visual and photographic observation of phenotype is made and recorded for each infected plant and each of its controls and a comparison is made. Data is entered into a Laboratory Information Management System database. At the end of the observation period, moderately stunted plants are grouped for analysis. The nucleic acid insert contained in the viral vector clone 740AT #2441 is responsible for moderate stunting of one of the plants. Clone 740AT #2441 is sequenced. The homolog from the plant host is also sequenced. The 740AT #2441 was sequenced and found to contain the Ran GTP binding protein cDNA sequence. The nucleic acid sequence homology between Arabidopsis and other plants exceeds 81%. The amino acid sequence homology between Arabidopsis and other plants exceeds 95%.

The present invention is also directed to a method of increasing yield of a grain crop. In Rice Biotechnology Quarterly (37:4, (1999)), it is reported that a transgenic rice plant transformed with a rgpl gene, which encodes a small GTP binding protein from rice, was shorter than a control plant, but it produced more seeds than the control plant. To increase the yield of a grain crop, the present method comprises expressing a nucleic acid sequence of a donor plant in a positive sense orientation in the cytoplasm of the grain crop, wherein said expressing results in stunted growth and increased seed production of said grain crop. A preferred method comprises the steps of cloning the nucleic acid sequence into a plant viral vector and infecting the grain crop with a recombinant viral nucleic acid comprising said nucleic acid sequence. Preferred plant viral vector is derived from a Brome Mosaic virus, a Rice Necrosis virus, or a geminivirus. Preferred grain crops include rice, wheat, and barley. The nucleic acid expressed in the host plant, for example, comprises a GTP binding protein open reading frame having a positive sense orientation. The present method provides a transiently expression of a gene to obtain a stunted plant. Because less energy is put into plant growth, more energy is available for production of seed, which results in increase yield of a grain crop. The present method has an advantage over other method using a trangenic plant, because it does not have an effect on the genome of a host plant.

In order to provide an even clearer and more consistent understanding of the specification and the claims, including the scope given herein to such terms, the following definitions are provided:

Adjacent: A position in a nucleotide sequence proximate to and 5′ or 3′ to a defined sequence. Generally, adjacent means within 2 or 3 nucleotides of the site of reference.

Anti-Sense Inhibition: A type of gene regulation based on cytoplasmic, nuclear or organelle inhibition of gene expression due to the presence in a cell of an RNA molecule complementary to at least a portion of the mRNA being translated. It is specifically contemplated that RNA molecules may be from either an RNA virus or mRNA from the host cells genome or from a DNA virus.

Cell Culture: A proliferating group of cells which may be in either an undifferentiated or differentiated state, growing contiguously or non-contiguously.

Chimeric Sequence or Gene: A nucleotide sequence derived from at least two heterologous parts. The sequence may comprise DNA or RNA.

Coding Sequence: A deoxyribonucleotide or ribonucleotide sequence which, when either transcribed and translated or simply translated, results in the formation of a cellular polypeptide or a ribonucleotide sequence which, when translated, results in the formation of a cellular polypeptide.

Compatible: The capability of operating with other components of a system. A vector or plant or animal viral nucleic acid which is compatible with a host is one which is capable of replicating in that host. A coat protein which is compatible with a viral nucleotide sequence is one capable of encapsidating that viral sequence.

Complementation Analysis: As used herein, this term refers to observing the changes produced in an organism when a nucleic acid sequence is introduced into that organism after a selected gene has been deleted or mutated so that it no longer functions fully in its normal role. A complementary gene to the deleted or mutated gene can restore the genetic phenotype of the selected gene.

Dual Heterologous Subgenomic Promoter Expression System (DHSPES): a plus stranded RNA vector having a dual heterologous subgenomic promoter expression system to increase, decrease, or change the expression of proteins, peptides or RNAs, preferably those described in U.S. Pat. Nos. 5,316,931, 5,811,653, 5,589,367, and 5,866,785, the disclosure of which is incorporated herein by reference.

Expressed sequence tags (ESTs): Relatively short single-pass DNA sequences obtained from one or more ends of cDNA clones and RNA derived therefrom. They may be present in either the 5′ or the 3′ orientation. ESTs have been shown useful for identifying particular genes.

Expression: The term as used herein is meant to incorporate one or more of transcription, reverse transcription and translation.

Gene: A discrete nucleic acid sequence responsible for producing one or more cellular products and/or performing one or more intercellular or intracellular functions.

Gene silencing: A reduction in gene expression. A viral vector expressing gene sequences from a host may induce gene silencing of homologous gene sequences.

A functional Gene Profile: The collection of genes of an organism which code for a biochemical or phenotypic trait. The functional gene profile of an organism is found by screening nucleic acid sequences from a donor organism by over expression or suppression of a gene in a host organism. A functional gene profile requires a collection or library of nucleic acid sequences from a donor organism. A functional gene profile will depend on the ability of the collection or library of donor nucleic acids to cause over-expression or suppression in the host organism. Therefore, a functional gene profile will depend upon the quantity of donor genes capable of causing over-expression or suppression of host genes or of being expressed in the host organism in the absence of a homologous host gene.

Homology: A degree of nucleic acid similarity in all or some portions of an endogenous gene sequence sufficient to result in suppression or overproduction of the endogenous gene when the nucleic acid sequence is delivered in a positive orientation.

Host: A cell, tissue or organism capable of replicating a nucleic acid such as a vector or plant viral nucleic acid and which is capable of being infected by a virus containing the viral vector or viral nucleic acid. This term is intended to include prokaryotic and eukaryotic cells, organs, tissues or organisms, where appropriate. Bacteria, fungi, yeast, animal (cell, tissues, or organisms), and plant (cell, tissues, or organisms) are examples of a host.

Infection: The ability of a virus to transfer its nucleic acid to a host or introduce a viral nucleic acid into a host, wherein the viral nucleic acid is replicated, viral proteins are synthesized, and new viral particles assembled. In this context, the terms “transmissible” and “infective” are used interchangeably herein. The term is also meant to include the ability of a selected nucleic acid sequence to integrate into a genome, chromosome or gene of a target organism.

Multigene family: A set of genes descended by duplication and variation from some ancestral gene. Such genes may be clustered together on the same chromosome or dispersed on different chromosomes. Examples of multigene families include those which encode the histones, hemoglobins, immunoglobulins, histocompatibility antigens, actins, tubulins, keratins, collagens, heat shock proteins, salivary glue proteins, chorion proteins, cuticle proteins, yolk proteins, and phaseolins.

Non-Native: Any RNA or DNA sequence that does not normally occur in the cell or organism in which it is placed. Examples include recombinant plant viral nucleic acids and genes or ESTs contained therein. That is, an RNA or DNA sequence may be non-native with respect to a viral nucleic acid. Such an RNA or DNA sequence would not naturally occur in the viral nucleic acid. Also, an RNA or DNA sequence may be non-native with respect to a host organism. That is, such a RNA or DNA sequence would not naturally occur in the host organism.

Nucleic acid: As used herein the term is meant to include any DNA or RNA sequence from the size of one or more nucleotides up to and including a complete gene sequence. The term is intended to encompass all nucleic acids whether naturally occurring in a particular cell or organism or non-naturally occurring in a particular cell or organism.

Phenotypic Trait: An observable, measurable or detectable property resulting from the expression or suppression of a gene or genes.

Plant Cell: The structural and physiological unit of plants, consisting of a protoplast and the cell wall.

Plant Organ: A distinct and visibly differentiated part of a plant, such as root, stem, leaf or embryo.

Plant Tissue: Any tissue of a plant in planta or in culture. This term is intended to include a whole plant, plant cell, plant organ, protoplast, cell culture, or any group of plant cells organized into a structural and functional unit.

Positive-sense inhibition: A type of gene regulation based on cytoplasmic inhibition of gene expression due to the presence in a cell of an RNA molecule substantially homologous to at least a portion of the mRNA being translated.

Promoter: The 5′-flanking, non-coding sequence substantially adjacent a coding sequence which is involved in the initiation of transcription of the coding sequence.

Recombinant Plant Viral Nucleic Acid: Plant viral nucleic acid which has been modified to contain non-native nucleic acid sequences. These non-native nucleic acid sequences may be from any organism or purely synthetic, however, they may also include nucleic acid sequences naturally occurring in the organism into which the recombinant plant viral nucleic acid is to be introduced.

Recombinant Plant Virus: A plant virus containing the recombinant plant viral nucleic acid.

Subgenomic Promoter: A promoter of a subgenomic mRNA of a viral nucleic acid.

Substantial Sequence Homology: Denotes nucleotide sequences that are substantially functionally equivalent to one another. Nucleotide differences between such sequences having substantial sequence homology are insignificant in affecting function of the gene products or an RNA coded for by such sequence.

Systemic Infection: Denotes infection throughout a substantial part of an organism including mechanisms of spread other than mere direct cell inoculation but rather including transport from one infected cell to additional cells either nearby or distant.

Transposon: A nucleotide sequence such as a DNA or RNA sequence which is capable of transferring location or moving within a gene, a chromosome or a genome.

Transgenic plant: A plant which contains a foreign nucleotide sequence inserted into either its nuclear genome or organellar genome.

Plant are grown from seed in a mixture of “Peat-Lite Mix™ (Speedling, Inc. Sun City, Fla.) and Nutricote™ controlled release fertilizer 14-14-14 (Chiss-Asahi Fertilizer Co., Tokyo, Japan). Plants are grown in a controlled environment provided 16 hours of light and 8 hours of darkness. Sylvania “Gro-Lux/Aquarium” wide spectrum 40 watt flourescent grow lights (Osram Sylvania Products, Inc., Danvers, Mass.) are used. Temperatures are kept at around 80° F. during light hours and 70° F. during dark hours. Humidity is between 60 and 85%.

Transcription: Production of an RNA molecule by RNA polymerase as a complementary copy of a DNA sequence or subgenomic mRNA.

Vector: A self-replicating RNA or DNA molecule which transfers an RNA or DNA segment between cells, such as bacteria, yeast, plant, or animal cells.

Virus: An infectious agent composed of a nucleic acid which may or may not be encapsidated in a protein. A virus may be a mono-, di-, tri-, or multi-partite virus, as described above.

### EXAMPLES

The following examples further illustrate the present invention. These examples are intended merely to be illustrative of the present invention and are not to be construed as being limiting.

### Example 1

Gene Silencing/Co-Supression of Genes Induced by Delivering an RNA Capable of Base Pairing with Itself to Form Double Stranded Regions.

Gene silencing has been used to down regulate gene expression in transgenic plants. Recent experimental evidence suggests that double stranded RNA may be an effective stimulator of gene silencing/co-suppression phenomenon in transgenic plant. For example, Waterhouse et al. (Proc. Natl. Acad. Sci. USA 95:13959-13964 (1998), incorporated herein by reference) described that virus resistance and gene silencing in plants could be induced by simultaneous expression of sense and antisense RNA. Gene silencing/co-suppression of plant genes may be induced by delivering an RNA capable of base pairing with itself to form double stranded regions.

This example shows: (1) a novel method for generating an RNA virus vector capable of producing an RNA capable of forming double stranded regions, and (2) a process to silence plant genes by using such a viral vector.

Step 1: Construction of a DNA sequence which after it is transcribed would generate an RNA molecule capable of base pairing with itself. Two identical, or nearly identical, ds DNA sequences are ligated together in an inverted orientation to each other (i.e., in either a head to tail or tail to head orientation) with or without a linking nucleotide sequence between the homologous sequences. The resulting DNA sequence is then be cloned into a cDNA copy of a plant viral vector genome.

Step 2: Cloning, screening, transcription of clones of interest using known methods in the art.

Step 3: Infect plant cells with transcripts from clones.

As virus expresses foreign gene sequence, RNA from foreign gene forms base pair upon itself, forming double-stranded RNA regions. This approach is used with any plant or non-plant gene and used to silence plant gene homologous to assist in identification of the function of a particular gene sequence.

### Example 2

Cytoplasmic Inhibition of Phytoene Desaturase in a Transfected Plant Confirms that the Partial Tomato PDS Sequence Encodes Phytoene Desaturase.

Isolation of tomato mosaic virus cDNA. An 861 base pair fragment (5524-6384) from the tomato mosaic virus (fruit necrosis strain F; tom-F) containing the putative coat protein subgenomic promoter, coat protein gene, and the 3′-end was isolated by PCR using primers

| | | |
|---|---|---|
| 5′-CTCGCAAAGTTTCGAACCAAATCCTC3′ | (upstream) and | (SEQ ID NO:1) |
| 5′-CGGGGTACCTGGGCCCCAACCGGGGGTTCCGGGGG-3′ | (downstream) | (SEQ ID NO:2) |

and subcloned into the HincII site of pbluescript KS-. A hybrid virus consisting of TMV-U1 and ToMV-F was constructed by swapping an 874-bp BamHI-KpnI ToMV fragment into pBGC152, creating plasmid TTO1. The inserted fragment was verified by dideoxynucleotide sequencing. A unique AvrII site was inserted downstream of the XhoI site in TTO1 by PCR mutagenesis, creating plasmid TTO1A, using the following oligonucleotides:

| | | |
|---|---|---|
| 5′-TCCTCGAGCCTAGGCTCGCAAAGTTTCGAACCAAATCCTCA-3′ | (upstream), | (SEQ ID NO:3) |
| 5′-CGGGGTACCTGGGCCCCAACCGGGGGTTCCGGGGG-3′ | (downstream). | (SEQ ID NO:4) |

Isolation of a cDNA encoding tomato phytoene synthase and a partial cDNA encoding tomato phytoene desaturase. Partial cDNAs were isolated from ripening tomato fruit RNA by polymerase chain reaction (PCR) using the following oligonucleotides:

| | | | |
|---|---|---|---|
| PSY, | 5′-TATGTATGGTGCAGAAGAACAGAT-3′ | (upstream), | (SEQ ID NO:5) |
| | 5′-AGTCGACTCTTCCTCTTCTGGCATC-3′ | (downstream); | (SEQ ID NO:6) |
| PDS, | 5′-TGCTCGAGTGTGTTCTTCAGTTTTCTGTCA-3′ | (upstream), | (SEQ ID NO:7) |
| | 5′-AACTCGAGCGCTTTGATTTCTCCGAAGCTT-3′ | (downstream). | (SEQ ID NO:8) |

Approximately 3×10⁴ colonies from a Lycopersicon esculentum cDNA library were screened by colony hybridization using a ³²P labeled tomato phytoene synthase PCR product. Hybridization was carried out at 42° C. for 48 hours in 50% formamide, 5×SSC, 0.02 M phosphate buffer, 5× Denhart's solution, and 0.1 mg/ml sheared calf thymus DNA. Filters were washed at 65° C. in 0.1×SSC, 0.1% SDS prior to autoradiography. PCR products and the phytoene synthase cDNA clones were verified by dideoxynucleotide sequencing.

DNA sequencing and computer analysis. A PstI, BamHI fragment containing the phytoene synthase cDNA and the partial phytoene desaturase cDNA was subcloned into pBluescript® KS+(Stratagene, La Jolla, Calif.). The nucleotide sequencing of KS+/PDS #38 and KS+/5′3′PSY was carried out by dideoxy termination using single-stranded templates (Maniatis, Molecular Cloning, 1^{st} Ed.) Nucleotide sequence analysis and amino acid sequence comparisons were performed using PCGENE® and DNA Inspector® IIE programs.

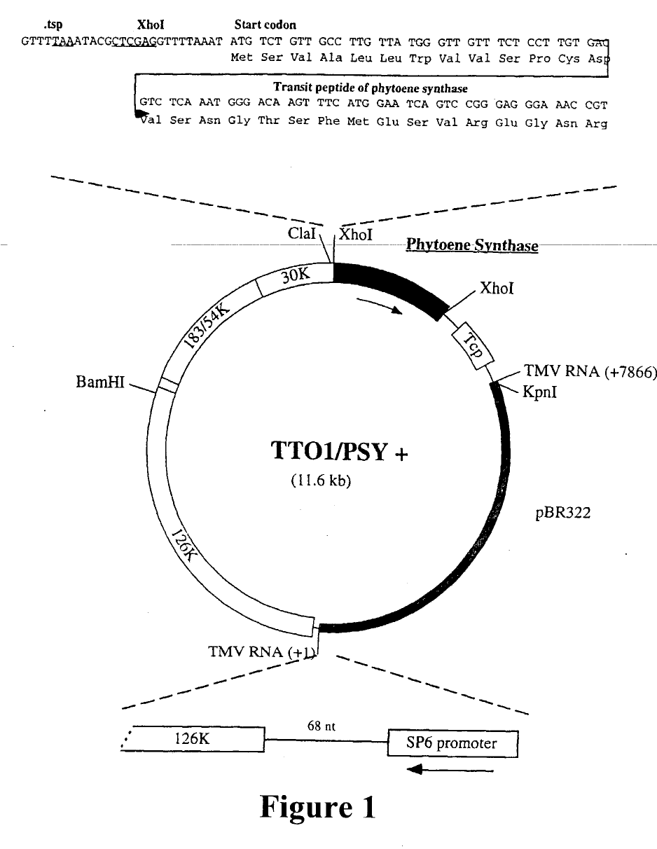

Construction of the tomato phytoene synthase expression vector. A XhoI fragment containing the tomato phytoene synthase cDNA was subcloned into TTO1. The vector TTO1/PSY+ (FIG. 1, SEQ ID NOs: 9 and 10) contains the phytoene synthase cDNA in the positive orientation under the control of the TMV-U1 coat protein subgenomic promoter; while, the vector TTO1/PSY− contains the phytoene synthase cDNA in the antisense orientation.

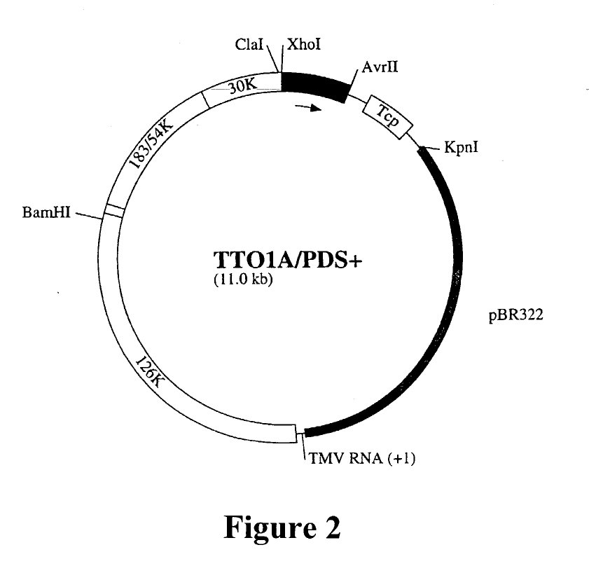

Construction of a viral vector containing a partial tomato phytoene desaturase cDNA. A XhoI fragment containing the partial tomato phytoene desaturase cDNA was subcloned into TTO1. The vector TTO1A/PDS+ (FIG. 2) contains the phytoene desaturase cDNA in the positive orientation under the control of the TMV-U1 coat protein subgenomic promoter; while the vector TTO1A/PDS− contains the phytoene desaturase cDNA in the antisense orientation.

Analysis of N. benthamiana transfected by TTO1/PSY+, TTO1I/PSY−, TTO1A/PDS+, TTO1/PDS−. Infectious RNAs from TTO1/PSY+, TTO1/PSY−, TTO1/PDS+, and TTO1/PDS−, were prepared by in vitro transcription using SP6 DNA-dependent RNA polymerase as described previously (Dawson et al., Proc. Natl. Acad. Sci. USA 85:1832 (1986)) and were used to mechanically inoculate N. benthamiana. The hybrid viruses spread throughout all the non-inoculated upper leaves as verified by transmission electron microscopy, local lesion infectivity assay, and polymerase chain reaction (PCR) amplification. The viral symptoms resulting from the infection consisted of distortion of systemic leaves and plant stunting with mild chlorosis. The leaves from plants transfected with TTO1/PSY+ turned orange and accumulated high levels of phytoene while those transfected with TTO1/PDS+ and TTO11PDS− turned white. Agarose gel eletrophoresis of PCR cDNA isolated from virion RNA and Northern blot analysis of virion RNA indicate that the vectors are maintained in an extrachromosomal state and have not undergone any detectable intramolecular rearrangements.

Purification and analysis of carotenoids from transfected plants. The carotenoids were isolated from systemically infected tissue and analyzed by HPLC chromatography. Carotenoids were extracted in ethanol and identified by their peak retention time and absorption spectra on a 25-cm Spherisorb® ODS-15-m column using acetonitrile/methanol/2-propanol (85:10:5) as a developing solvent at a flow rate of 1 m/min. They had identical retention time to a synthetic phytoene standard and β-carotene standards from carrot and tomato. The phytoene peak from N. benthamiana transfected with TTO1/PSY+ had an optical absorbance maxima at 276, 285, and 298 nm. Plants transfected with viral encoded phytoene synthase showed a ten-fold increase in phytoene compared to the levels in noninfected plants. The expression of sense and antisense RNA to a partial phytoene desaturase in transfected plants increased the level of phytoene and altered the biochemical pathway; it thus inhibited the synthesis of colored carotenoids and caused the systemically infected leaves to turn white. HPLC analysis of these plants revealed that they also accumulated phytoene. The white leaf phenotype was also observed in plants treated with the herbicide norflurazon which specifically inhibits phytoene desaturase.

This change in the levels of phytoene represents one of the largest increases of any carotenoid (secondary metabolite) in any genetically engineered plant. Plants transfected with viral-encoded phytoene synthase in a plus sense showed a ten-fold increase in phytoene compared to the levels in noninfected plants. In addition, the accumulation of phytoene in plants transfected with antisense phytoene desaturase suggests that viral vectors can be used as a potent tool to manipulate pathways in the production of secondary metabolites through cytoplasmic antisense inhibition. Leaves from systemically infected TTO1A/PDS+ plants also accumulated phytoene and developed a bleaching white phenotype; the actual mechanism of inhibition is not clear. These data are presented by Kumagai et al., Proc. Natl. Acad. Sci. USA 92:1679-1683 (1995).

### Example 3

Expression of Bell Pepper cDNA in Transfected Plant Confirms that it Encodes Capsanthin-Capsorubin Synthase.

The biosynthesis of leaf carotenoids in Nicotiana benthamiana was altered by rerouting the pathway to the synthesis of capsanthin, a non-native chromoplast-specific xanthophyll, using an RNA viral vector. A cDNA encoding capsanthin-capsorubin synthase (Ccs), was placed under the transcriptional control of a tobamovirus subgenomic promoter. Leaves from transfected plants expressing Ccs developed an orange phenotype and accumulated high levels of capsanthin. This phenomenon was associated by thylakoid membrane distortion and reduction of grana stacking. In contrast to the situation prevailing in chromoplasts, capsanthin was not esterified and its increased level was balanced by a concomitant decrease of the major leaf xanthophylls, suggesting an autoregulatory control of chloroplast carotenoid composition. Capsanthin was exclusively recruited into the trimeric and monomeric light-harvesting complexes of Photosystem II. This demonstration that higher plant antenna complexes can accommodate non-native carotenoids provides compelling evidence for functional remodeling of photosynthetic membranes by rational design of carotenoids.

Construction of the Ccs expression vector. Unique XhoI, AvrII sites were inserted into the bell pepper capsanthin-capsorubin synthase (Ccs) cDNA by polymerase chain reaction (PCR) mutagenesis using oligonucleotides:

| | | |
|---|---|---|
| 5′-GCCTCGAGTGCAGCATGGAAACCCTTCTAAAGCTTTTCC-3′ | (upstream), | (SEQ ID NO:11) |
| 5′-TCCCTAGGTCAAAGGCTCTCTATTGCTAGATTGCCC-3′ | (downstream). | (SEQ ID NO:12) |

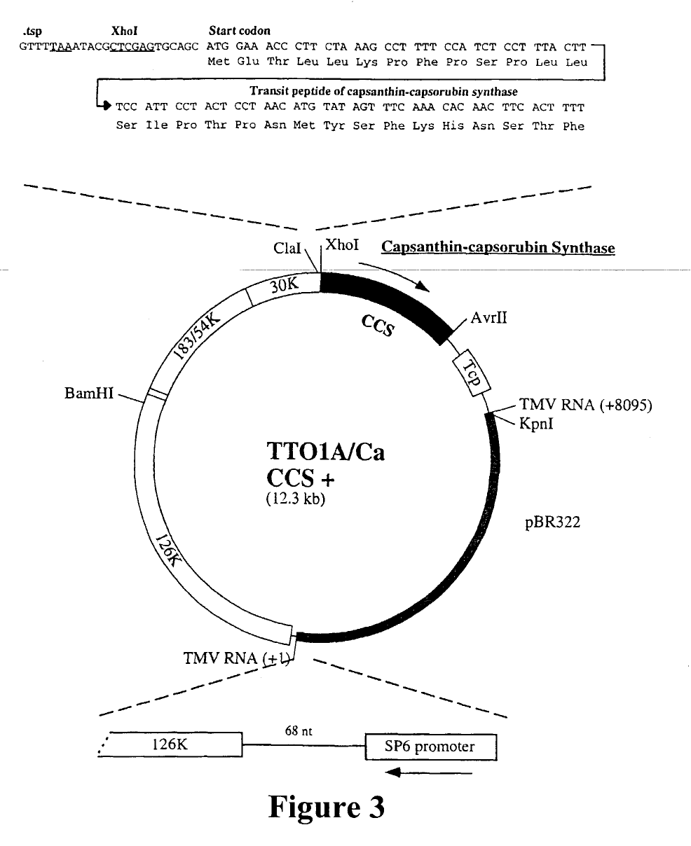

The 1.6-kb XhoI, AvrII cDNA fragment was placed under the control of the TMV-Ul coat protein subgenomic promoter by subcloning into TTO1A, creating plasmid TTO1A CCS+ (FIG. 3) in the sense orientation as represented by FIG. 3.

Carotenoid analysis. Twelve days after inoculation upper leaves from 12 plants were harvested and lyophilized. The resulting non-saponified extract was evaporated to dryness under argon and weighed to determine the total lipid content. Pigment analysis from the total lipid content was performed by HPLC and also separated by thin layer chromatography on silica gel G using hexane/acetone (60:40 (V/V)). Plants transfected with TTO1A CCS+ accumulated high levels of capsanthin (36% of total carotenoids).

### Example 4

Expression of Chinese Cucumber cDNA Clone pQ21D in Transfected Plants Confirms that it Encodes α-Trichosanthin.

We have developed a plant viral vector that directs the expression of α-trichosanthin in transfected plants. The open reading frame (ORF) for α-trichosanthin, from the genomic clone SEO, was placed under the control of the TMV coat protein subgenomic promoter. Infectious RNA from TTU51A QSEO #3 (FIG. 3) was prepared by in vitro transcription using SP6 DNA-dependent RNA polymerase and was used to mechanically inoculate N. benthamiana. The hybrid virus spread throughout all the non-inoculated upper leaves as verified by local lesion infectivity assay, and PCR amplification. The viral symptoms consisted of plant stunting with mild chlorosis and distortion of systemic leaves. The 27-kDa α-trichosanthin accumulated in upper leaves (14 days after inoculation) and crossreacted with an anti-trichosanthin antibody.

Plasmid Constructions.

An 0.88-kb XhoI, AvrII fragment, containing the α-trichosanthin coding sequence, was amplified from genomic DNA isolated from Trichosanthes kirilowii Maximowicz by PCR mutagenesis using oligonucleotides QMIX:

| (SEQ ID NO:13) | | |
|---|---|---|
| QMIX: | 5′-GCC TCG AGT GCA GCA TGA TCA GAT TCT TAG TCC TCT CTT TGC-3′ | (upstream) and |
| (SEQ ID NO:14) | | |
| Q1266A | 5′-TCC CTA GGC TAA ATA GCA TAA CTT CCA CAT CA AAGC-3′ | (downstream). |

The α-trichosanthin open reading frame was verified by dideoxy sequencing, and placed under the control of the TMV-U1 coat protein subgenomic promoter by subcloning into TTU51A, creating plasmid TTU51A QSEO #3.

In vitro Transcriptions, Inoculations, and Analysis of Transfected Plants.

N. benthaminana plants were inoculated with in vitro transcripts of Kpn I-digested TTU51A QSEO #3 as previously described (Dawson et al., supra). Virions were isolated from N. benthamiana leaves infected with TTU51A QSEO #3 transcripts.

Purification, Immunological Detection, and in vitro Assay of α-Trichosanthin.

Two weeks after inoculation, total soluble protein was isolated from upper, noninoculated N. benthamiana leaf tissue and assayed from cross-reactivity to a α-trichosanthin antibody. The proteins from systemically infected tissue were analyzed on a 0.1% SDS/12.5% polyacrylamide gel and transferred by electroblotting for 1 hr to a nitrocellulose membrane. The blotted membrane was incubated for 1 hr with a 2000-fold dilution of goat anti-α-trichosanthin antiserum. The enhanced chemiluminescence horseradish peroxidase-linked, rabbit anti-goat IgG assay (Cappel Laboratories) was performed according to the manufacturer's (Amersham) specifications. The blotted membrane was subjected to film exposure times of up to 10 sec. Shorter and longer chemiluminescent exposure times of the blotted membrane gave the same quantitative results.

### Example 5

Construction of a Tobamoviral Vector for Expression of Heterologous Genes in A. thaliana.

Virions that were prepared as a crude aqueous extract of tissue from turnip infected with Ribgrass mosaic virus (RMV) were used to inoculate N. benthamiana, N. tabacum, A. thaliana, and oilseed rape (canola). Two to three weeks after transfection, systemically infected plants were analyzed by immunoblotting, using purified RMV as a standard. Total soluble plant protein concentrations were determined using bovine serum albumin as a standard. The proteins were analyzed on a 0.1% SDS/12.5% polyacrylamide gel and transferred by electroblotting for 1 hr to a nitrocellulose membrane. The blotted membrane was incubated for 1 hr with a 2000-fold dilution of anti-ribgrass mosaic virus coat antiserum. Using standard protocols, the antisera was raised in rabbits against purified RMV coat protein. The enhanced chemiluminescence horseradish peroxidase-linked, goat anti-rabbit IgG assay (Cappel Laboratories) was performed according to the manufacturer's (Amersham) specifications. The blotted membrane was subjected to film exposure times of up to 10 sec. No detectable cross-reacting protein was observed in the noninfected N. benthamiana control plant extracts. A 18 kDa protein cross-reacted to the anti-RMV coat antibody from systemically infected N. benthamiana, N. tabacum, A. thaliana, and oilseed rape (canola). This result demonstrates that RMV can systemically infect N. benthamiana, N. tabacum, A. thaliana, and oilseed rape (canola).

Plasmid Constructions.

Ribgrass mosaic virus (RMV) is a member of the tobamovirus group that infects crucifers. A partial RMV cDNA containing the 30K subgenomic promoter, 30K ORF, coat subgenomic promoter, coat ORF, and 3′-end was isolated by RT-PCR using oligonucleotides

| (SEQ ID NO:15) | | |
|---|---|---|
| TVCV183X, | 5′-TAC TCG AGG TTC ATA AGA CCG CGG TAG GCG G-3′ | (upstream) and |
| (SEQ ID NO:16) | | |
| TVCV KpnI, | 5′-CGG GGT ACC TGG GCC CCT ACC CGG GGT TTA GGG AGG-3′ | (downstream), |

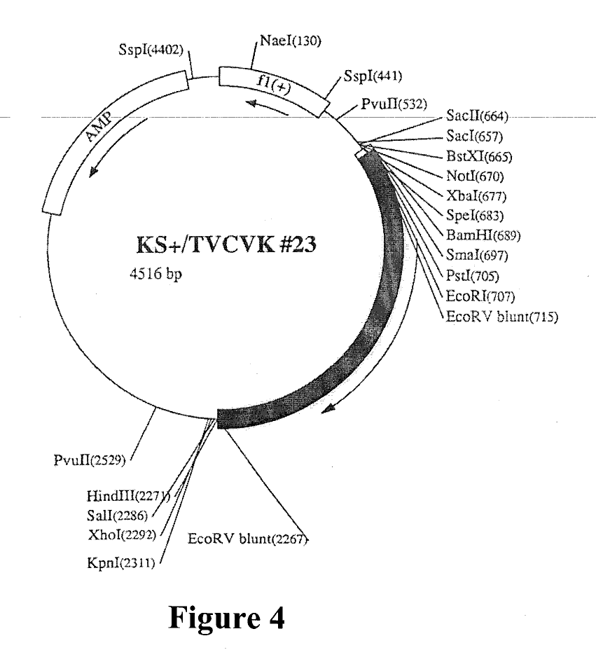

and subcloned into the EcoRV site of KS+, creating plasmid KS+ TVCV #23 (FIG. 4). The RMV cDNA was characterized by restriction mapping and dideoxy nucleotide sequencing. The partial nucleotide sequence is as follows:

| | |
|---|---|
| 5′-CTCGAGGTTCATAAGACCGCGGTAGGCGGAGCGTTTGTTTACTGTAGTATAATT | (SEQ ID NO:17) |
| AAATATTTGTCAGATAAAAGGTTGTTTAAAGATTTGTTTTTTGTTTGACTGAGTC | |
| GATAATGTCTTACGAGCCTAAAGTTAGTGACTTCCTTGCTCTTACGAAAAAGGA | |
| GGAAATTTTACCCAAGGCTTTGACGAGATTAAAGACTGTCTCTATTAGTACTAA | |
| GGATGTTATATCTGTTAAGGAGTCTGAGTCCCTGTGTGATATTGATTTGTTAGTG | |
| AATGTGCCATTAGATAAGTATAGGTATGTGGGTGTTTTGGGTGTTGTTTTCACCG | |
| GTGAATGGCTGGTACCGGATTTCGTTAAAGGTGGGGTAACAGTGAGCGTGATTG | |
| ACAAACGGCTTGAAAATTCCAGAGAGTGCATAATTGGTACGTACCGAGCTGCTG | |
| TAAAGGACAGAAGGTTCCAGTTCAAGCTGGTTCCAAATTACTTCGTATCCATTG | |
| CGGATGCCAAGCGAAAACCGTGGCAGGTTCATGTGCGAATTCAAAATCTGAAG | |
| ATCGAAGCTGGATGGCAACCTCTAGCTCTAGAGGTGGTTTCTGTTGCCATGGTTA | |
| CTAATAACGTGGTTGTTAAAGGTTTGAGGGAAAAGGTCATCGCAGTGAATGATC | |
| CGAACGTCGAAGGTTTCGAAGGTGTGGTTGACGATTTCGTCGATTCGGTTGCTG | |
| CATTCAAGGCGATTGACAGTTTCCGAAAGAAAAAGAAAAAGATTGGAGGAAGG | |
| GATGTAAATAATAATAAGTATAGATATAGACCGGAGAGATACGCCGGTCCTGAT | |
| TCGTTACAATATAAAGAAGAAAATGGTTTACAACATCACGAGCTCGAATCAGTA | |
| CCAGTATTTCGCAGCGATGTGGGCAGAGCCCACAGCGATGCTTAACCAGTGCGT | |
| GTCTGCGTTGTCGCAATCGTATCAAACTCAGGCGGCAAGAGATACTGTTAGACA | |
| GCAGTTCTCTAACCTTCTGAGTGCGATTGTGACACCGAACCAGCGGTTTCCAGA | |
| AACAGGATACCGGGTGTATATTAATTCAGCAGTTCTAAAACCGTTGTACGAGTC | |
| TCTCATGAAGTCCTTTGATACTAGAAATAGGATCATTGAAACTGAAGAAGAGTC | |
| GCGTCCATCGGCTTCCGAAGTATCTAATGCAACACAACGTGTTGATGATGCGAC | |
| CGTGGCCATCAGGAGTCAAATTCAGCTTTTGCTGAACGAGCTCTCCAACGGACA | |
| TGGTCTGATGAACAGGGCAGAGTTCGAGGTTTTATTACCTTGGGCTACTGCGCC | |
| AGCTACATAGGCGTGGTGCACACGATAGTGCATAGTGTTTTTCTCTCCACTTAAA | |
| TCGAAGAGATATACTTACGGTGTAATTCCGCAAGGGTGGCGTAAACCAAATTAC | |
| GCAATGTTTTAGGTTCCATTTAAATCGAAACCTGTTATTTCCTGGATCACCTGTT | |
| AACGTACGCGTGGCGTATATTACAGTGGGAATAACTAAAAGTGAGAGGTTCGA | |
| ATCCTCCCTAACCCCGGGTAGGGGCCCA-3′. | |

The 1543 base pair from the partial RMV cDNA was compared (PCGENE) to oilseed rape mosaic virus (ORMV). The nucleotide sequence identity was 97.8%. The RMV 30K and coat ORF were compared to ORMV and the amino acid identity was 98.11% (30K) and 98.73% (coat), respectively. A partial RMV cDNA containing the 5′-end and part of the replicase was isolated by RT-PCR from RMV RNA using oligonucleotides

(SEQ ID NO:18)

| | | |
|---|---|---|
| RGMV1 | 5′-GAT GGC GCC TTA ATA CGA CTC ACT ATA GTT TTA TTT TTG TTG CAA CAA CAA CAA C-3′ | (upstream) and |

(SEQ ID NO:19)

| | | |
|---|---|---|
| RGR 132 | 5′-CTT GTG CCC TTC ATG ACG AGC TAT ATC ACG-3′ | (downstream). |

The RMV cDNA was characterized by dideoxy nucleotide sequencing. The partial nucleotide sequence containing the T7 RNA polymerase promoter and part of the RMV cDNA is as follows:

| | |
|---|---|
| 5′-ccttaatacgactcactataGTTTTATTTTTGTTGCAACAACAACAACAAATTACAATAACAA | (SEQ ID NO:20) |
| CAAAACAAATACAAACAACAACAACATGGCACAATTTCAACAAACAGTAAACA | |
| TGCAAACATTGCAGGCTGCCGCAGGGCGCAACAGCCTGGTGAATGATTTAGCCT | |
| CACGACGTGTTTATGACAATGCTGTCGAGGAGCTAAATGCACGCTCGAGACGCC | |
| CTAAGGTTCATTACTCCAAATCAGTGTCTACGGAACAGACGCTGTTAGCTTCAA | |
| ACGCTTATCCGGAGTTTGAGATTTCCTTTACTCATACCCAACATGCCGTACACTC | |
| CCTTGCGGGTTGGCCTAAGGACTCTTGAGTTAGAGTATCTCATGATGCAAGTTCC | |
| GTTCGGTTCTCTGACGTACGACATCGGTGGTAACTTTGCAGCGCACCTTTTCAAA | |
| GGACGCGACTACGTTCACTGCTGTATGCCAAACTTGGATGTACGTGATATAGCT-3′. | |

The uppercase letters are nucleotide sequences from RMV cDNA.

The lower case letters are nucleotide sequences from T7 RNA polymerase promoter. The nucleotide sequences from the 5′ and 3′ oligonucleotides are underlined.

Full-length infectious RMV cDNA clones were obtained by RT-PCR from RMV RNA using oligonucleotides

(SEQ ID NO:21)

| | | |
|---|---|---|
| RGMV1, | 5′-GAT GGC GCC TTA ATA CGA CTC ACT ATA GTT TTA TTT TTG TTG CAA CAA CAA CAA C-3′ | (upstream) and |

(SEQ ID NO:22)

| | | |
|---|---|---|
| RG1 APE, | 5′-ATC GTT TAA ACT GGG CCC CTA CCC GGG GTT AGG GAG G-3′ | (downstream). |

The RMV cDNA was characterized by dideoxy nucleotide sequencing. The partial nucleotide sequence containing the T7 RNA polymerase promoter and part of the RMV cDNA is as follows:

| | |
|---|---|
| 5′-CCTTAATACGACTCACTATAGTTTTATTTTTGTTGCAACAACAACAACAAATTAC | (SEQ ID NO:23) |
| AATAACAACAAAACAAATACAAACAACAACAACATGGCACAATTTCAACAAAC | |
| AGTAAACATGCAAACATTCCAGGCTGCCGCAGGGCGCAACAGCCTGGTGAATG | |
| ATTTAGCCTCACGACGTGTTTATGACAATGCTGTCGAGGAGCTAAATGCACGCT | |
| CGAGACGCCCTAAGGTTCATTACTCCAAATCAGTGTCTACGGAACAGACGCTGT | |
| TAGCTTCAAACGCTTATCCGGAGTTTGAGATTTCCTTTACTCATACCCAAACATG | |
| CCGTACACTCCCTTGCGGGTGGCCTAAGGACTCTTGAGTTAGAGTATCTCATGAT | |
| GCAAGTTCCGTTCGGTTCTCTGACGTACGACATCGGTGGTAACTTTGCAGCGCAC | |
| CTTTTCAAAGGACGCGACTACGTTCACTGCTGTATGCCAAACTTGGATGTACGTG | |
| ATATAGCT-3′ | |

The uppercase letters are nucleotide sequences from RMV cDNA. The nucleotide sequences from the 5′ and 3′ oligonucleotides are underlined. Full length infectious RMV cDNA clones were obtained by RT-PCR from RMV RNA using oligonucleotides

| | |
|---|---|
| RGMV1, | |
| 5′-gat ggc gcc tta ata cga ctc act ata gtt tta ttt ttg ttg caa caa caa caa c-3′ | (SEQ ID NO:24) |
| (upstream) and | |
| RG1 APE, | |
| 5′-ATC GTT TAA ACT GGG CCC CTA CCC GGG GTT AGG GAG G-3′ | (SEQ ID NO:25) |
| (downstream). | |

### Example 6

Arabidopsis thaliana cDNA Library Construction in a Dual Subgenomic Promoter Vector.

Arabidopsis thaliana cDNA libraries obtained from the Arabidopsis Biological Resource Center (ABRC). The four libraries from ABRC were size-fractionated with inserts of 0.5-1 kb (CD4-13), 1-2 kb (CD4-14), 2-3 kb (CD4-15), and 3-6 kb (CD4-16). All libraries are of high quality and have been used by several dozen groups to isolate genes. The pBluescript® phagemids from the Lambda ZAP II vector were subjected to mass excision and the libraries were recovered as plasmids according to standard procedures.

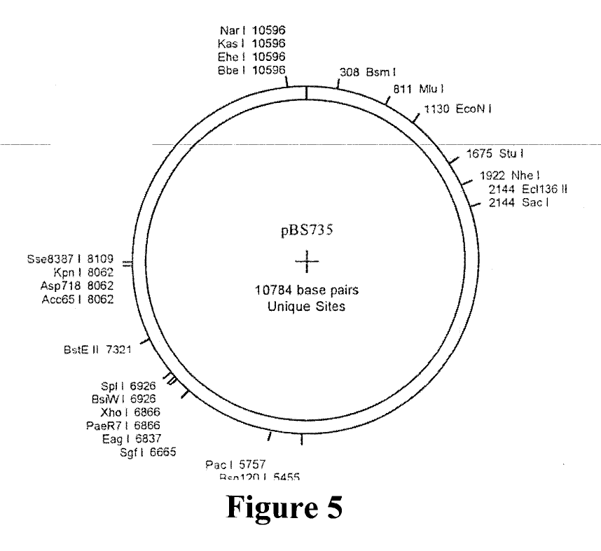

Alternatively, the cDNA inserts in the CD4-13 (Lambda ZAP II vector) were recovered by digestion with NotI. Digestion with NotI in most cases liberated the entire Arabidopsis thaliana cDNA insert because the original library was assembled with NotI adapters. NotI is an 8-base cutter that infrequently cleaves plant DNA. In order to insert the NotI fragments into a transcription plasmid, the pBS735 transcription plasmid (FIG. 5) was digested with PacIXhoI and ligated to an adapter DNA sequence created from the oligonucleotides

| | |
|---|---|
| 5′-TCGAGCGGCCGCAT-3′ and | (SEQ ID NO:26) |
| 5′-GCGGCCGC-3′. | (SEQ ID NO:27) |

The resulting plasmid pBS740 (FIG. 6) contains a unique NotI restriction site for bidirectional insertion of NotI fragments from the CD4-13 library. Recovered colonies were prepared from these for plasmid minipreps with a Qiagen BioRobot 9600®. The plasmid DNA preps performed on the BioRobot 9600® are done in 96-well format and yield transcription quality DNA. An Arabidopsis cDNA library was transformed into the plasmid and analyzed by agarose gel electrophoresis to identify clones with inserts. Clones with inserts are transcribed in vitro and inoculated onto N. benthamiana or Arabidopsis thaliana. Selected leaf disks from transfected plants are then taken for biochemical analysis.

### Example 7

High Throughput Robotics.

The efficiency of inoculation of subject organisms such as plants is improved by using means of high throughput robotics. For example, host plants such as Arabidopsis thaliana were grown in microtiter plates such as the standard 96-well and 384-well microtiter plates. A robotic handling arm then moved the plates containing the organism to a colony picker or other robot that delivered inoculations to each plant in the well. By this procedure, inoculation was performed in a very high speed and high throughput manner. It is preferable that the plant is a germinating seed or at least in the development cycle to enable access to the cells to be transfected. Equipments used for automated robotic production line include, but not be limited to, robots of these types: electronic multichannel pipetmen, Qiagen BioRobot 9600®, Robbins Hydra liquid handler, Flexys Colony Picker, New Brunswick automated plate pourer, GeneMachines HiGro shaker incubator, New Brunswick floor shaker, three Qiagen BioRobots, MJ Research PCR machines (PTC-200, Tetrad), ABI 377 sequencer and Tecan Genesis RSP200 liquid handler.

### Example 8

Genomic DNA Library Construction in a Recombinant Viral Nucleic Acid Vector.

Genomic DNAs represented in BAC (bacterial artificial chromosome) or YAC (yeast artificial chromosome) libraries are obtained from the Arabidopsis Biological Resource Center (ABRC). The BAC/YAC DNAs are mechanically size-fractionated, ligated to adapters with cohesive ends, and shotgun-cloned into recombinant viral nucleic acid vectors. Alternatively, mechanically size-fractionated genomic DNAs are blunt-end ligated into a recombinant viral nucleic acid vector. Recovered colonies are prepared for plasmid minipreps with a Qiagen BioRobot 9600®. The plasmid DNA preps done on the BioRobot 9600® are assembled in 96-well format and yield transcription quality DNA. The recombinant viral nucleic acid/Arabidopsis genomic DNA library is analyzed by agarose gel electrophoresis (template quality control step) to identify clones with inserts. Clones with inserts are then transcribed in vitro and inoculated onto N. benthamiana and/or Arabidopsis thaliana. Selected leaf disks from transfected plants are then be taken for biochemical analysis.

Genomic DNA from Arabidopsis typically contains a gene every 2.5 kb (kilobases) on average. Genomic DNA fragments of 0.5 to 2.5 kb obtained by random shearing of DNA were shotgun assembled in a recombinant viral nucleic acid expression/knockout vector library. Given a genome size of Arabidopsis of approximately 120,000 kb, a random recombinant viral nucleic acid genomic DNA library would need to contain minimally 48,000 independent inserts of 2.5 kb in size to achieve IX coverage of the Arabidopsis genome. Alternatively, a random recombinant viral nucleic acid genomic DNA library would need to contain minimally 240,000 independent inserts of 0.5 kb in size to achieve 1× coverage of the Arabidopsis genome. Assembling recombinant viral nucleic acid expression/knockout vector libraries from genomic DNA rather than cDNA has the potential to overcome known difficulties encountered when attempting to clone rare, low-abundance mRNA's in a cDNA library. A recombinant viral nucleic acid expression/knockout vector library made with genomic DNA would be especially useful as a gene silencing knockout library. In addition, the Dual Heterologous Subgenomic Promoter Expression System (DHSPES) expression/knockout vector library made with genomic DNA would be especially useful for expression of genes lacking introns. Furthermore, other plant species with moderate to small genomes (e.g. rose, approximately 80,000 kb) would be especially useful for recombinant viral nucleic acid expression/knockout vector libraries made with genomic DNA. A recombinant viral nucleic acid expression/knockout vector library can be made from existing BAC/YAC genomic DNA or from newly-prepared genomic DNAs for any plant species.

### Example 9

Genomic DNA or cDNA Library Construction in a DHSPES Vector, and Transfection of Individual Clones from said Vector Library onto T-DNA Tagged or Transposon Tagged or Mutated Plants.

Genomic DNA or cDNA library construction in a recombinant viral nucleic acid vector, and transfection of individual clones from the vector library onto T-DNA tagged or transposon tagged or mutated plants may be performed according the procedure set forth in Example 6. Such a protocol may be easily designed to complement mutations introduced by random insertional mutagenesis of T-DNA sequences or transposon sequences.

### Example 10

Production of a Malarial CTL Epitope Genetically Fused to the C Terminus of the TMVCP.

Malarial immunity induced in mice by irradiated sporozites of P. yoelii is also dependent on CD8+ T lymphocytes. Clone B is one ocytotoxic T lymphocyte (CTL) cell clone shown to recognize an epitope present in both the P. yoelii and P. berghei CS proteins. Clone B recognizes the following amino acid sequence; SYVPSAEQILEFVKQISSQ (SEQ ID NO: 28) and when adoptively transferred to mice, it protects against infection from both species of malaria sporozoites. Construction of a genetically modified tobamovirus designed to carry this malarial CTL epitope fused to the surface of virus particles is set forth herein. Construction of plasmid pBGC289. A 0.5 kb fragment of pBGC11 was PCR amplified using the 5′ primer TB2ClaI5′ and the 3′ primer C/-5AvrII. The amplified product was cloned into the Smal site of pBstKS+ (Stratagene Cloning Systems) to form pBGC214.

PBGC215 was formed by cloning the 0.15 kb AccI-NsiI fragment of pBGC214 into pBGC235. The 0.9 kb NcoI-KpnI fragment from pBGC215 was cloned in pBGC152 to form pBGC216.

A 0.07 kb synthetic fragment was formed by annealing PYCS.2p with PYCS.2m and the resulting double stranded fragment, encoding the P. yoelii CTL malarial epitope, was cloned into the AvrII site of pBGC215 made blunt ended by treatment with mung bean nuclease and creating a unique AatII site, to form pBGC262. A 0.03 kb synthetic AatII fragment was formed by annealing TLS.1EXP with TLS.1EXM, and the resulting double stranded fragment, encoding the leaky-stop sequence and a stuffer sequence used to facilitate cloning, was cloned into AatII digested pBGC262 to form pBGC263. PBGC262 was digested with AatII and ligated to itself removing the 0.02 kb stuffer fragment to form pBGC264. The 1.0 kb NcoI-KpnI fragment of pBGC264 was cloned into pSNC004 to form pBGC289.

The virus TMV289 produced by transcription of plasmid pBGC289 in vitro contains a leaky stop signal resulting in the removal of four amino acids from the C terminus of the wild type TMV coat protein gene and is therefore predicted to synthesize a truncated coat protein and coat protein with a CTL epitope fused at the C terminus at a ratio of 20:1. The recombinant TMVCP/CTL epitope fusion present in TMV289 is with the stop codon decoded as the amino acid Y (amino acid residue 156). The amino acid sequence of the coat protein of virus TMV216 produced by transcription of the plasmid pBGC216 in vitro, is truncated by four amino acids. The epitope SYVPSAEQILEFVKQISSQ is calculated to be present at approximately 0.5% of the weight of the virion using the same assumptions confirmed by quantitative ELISA analysis.

Propagation and purification of the epitope expression vector. Infectious transcripts were synthesized from KpnI-linearized pBGC289 using T7 RNA polymerase and cap (7mGpppG) according to the manufacturer (New England Biolabs).

An increased quantity of recombinant virus was obtained by passaging Sample ID No. TMV289.11B1a. Fifteen tobacco plants were grown for 33 days post inoculation accumulating 595 g fresh weight of harvested leaf biomass not including the two lower inoculated leaves. Purified Sample ID No. TMV289.11B2 was recovered (383 mg) at a yield of 0.6 mg virion per gram of fresh weight. Therefore, 3 g of 19-mer peptide was obtained per gram of fresh weight extracted. Tobacco plants infected with TMV289 accumulated greater than 1.4 micromoles of peptide per kilogram of leaf tissue.

Product analysis. Partial confirmation of the sequence of the epitope coding region of TMV289 was obtained by restriction digestion analysis of PCR amplified cDNA using viral RNA isolated from Sample ID No. TMV289.11B2. The presence of proteins in TMV289 with the predicted mobility of the cp fusion at 20 kD and the truncated cp at 17.1 kD was confirmed by denaturing polyacrylamide gel electrophoresis.

### Example 11

Identification of Nucleotide Sequences Involved in the Regulation of Plant Growth by Cytoplasmic Inhibition of Gene Expression Using Viral Derived RNA.

In the following examples, we show: (1) a method for producing plus sense RNA using an RNA viral vector, (2) a method to produce viral-derived sense RNA in the cytoplasm, (3) a method to enhance or suppress the expression of endogenous plant proteins in the cytoplasm by viral antisense RNA, and (4) a method to produce transfected plants containing viral plus sense RNA; such methods are much faster than the time required to obtain genetically engineered sense transgenic plants. Systemic infection and expression of viral plus sense RNA occurs as short as four days post inoculation, whereas it takes several months or longer to create a single transgenic plant. These examples demonstrates that novel positive strand viral vectors, which replicate solely in the cytoplasm, can be used to identify genes involved in the regulation of plant growth by enhancing or inhibiting the expression of specific endogenous genes. These examples enable one to characterize specific genes and biochemical pathways in transfected plants using an RNA viral vector.

Tobamoviral vectors have been developed for the heterologous expression of uncharacterized nucleotide sequences in transfected plants. A partial Arabidopsis thaliana cDNA library was placed under the transcriptional control of a tobamovirus subgenomic promoter in a RNA viral vector. Colonies from transformed E. coli were automatically picked using a Flexys robot and transferred to a 96 well flat bottom block containing terrific broth (TB) Amp 50 ug/ml. Approximately 2000 plasmid DNAs were isolated from overnight cultures using a BioRobot and infectious RNAs from 430 independent clones were directly applied to plants. One to two weeks after inoculation, transfected Nicotiana benthamiana plants were visually monitored for changes in growth rates, morphology, and color. One set of plants transfected with 740 AT #2441 were severely stunted. DNA sequence analysis revealed that this clone contained an Arabidopsis Ran GTP binding protein open reading frame (ORF) in a plus sense orientation. This demonstrates that an episomal RNA viral vector can be used to deliberately alter the metabolic pathway and cause plant stunting. In addition, our results show that the Arabidopsis plus sense transcript can cause phenotypic changes in N. benthamiana.

Construction of an Arabidopsis thaliana cDNA Library in an RNA Viral Vector.

An Arabidopsis thaliana CD4-13 cDNA library was digested with NotI. DNA fragments between 500 and 1000 bp were isolated by trough elution and subcloned into the NotI site of pBS740. E. coli C600 competent cells were transformed with the pBS740 AT library and colonies containing Arabidopsis cDNA sequences were selected on LB Amp 50 ug/ml. Recombinant C600 cells were automatically picked using a Flexys robot and then transferred to a 96 well flat bottom block containing terrific broth (TB) Amp 50 ug/ml. Approximately 2000 plasmid DNAs were isolated from overnight cultures using a BioRobot (Qiagen) and infectious RNAs from 430 independent clones were directly applied to plants.

Isolation of a Gene Encoding a GTP Binding Protein.

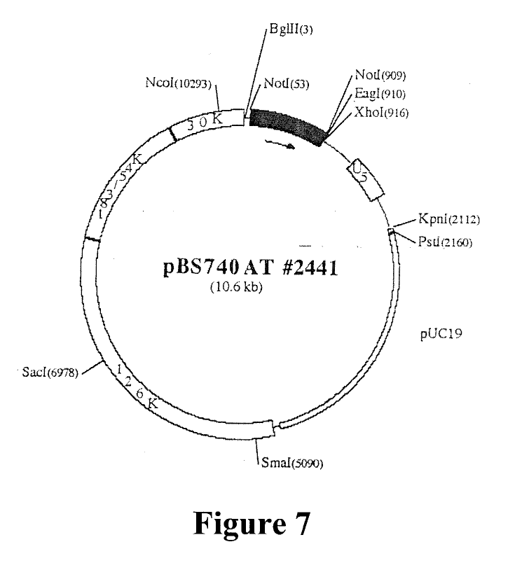

One to two weeks after inoculation, transfected Nicotiana benthamiana plants were visually monitored for changes in growth rates, morphology, and color. Plants transfected with 740 AT #2441 (FIG. 7) were severely stunted. Plasmid 740 AT #2441 contains the TMV-U1 open reading frames (ORFs) encoding 126-, 183-, and 30-kDa proteins, the TMV-U5 coat protein gene (U5 cp), the T7 promoter, an Arabidopsis thaliana CD4-13 NotI fragment, and part of the pUC19 plasmid. The TMV-U1 subgenomic promoter located within the minus strand of the 30-kDa ORF controls the synthesis of the CD4-13 subgenomic RNA.

DNA Sequencing and Computer Analysis.

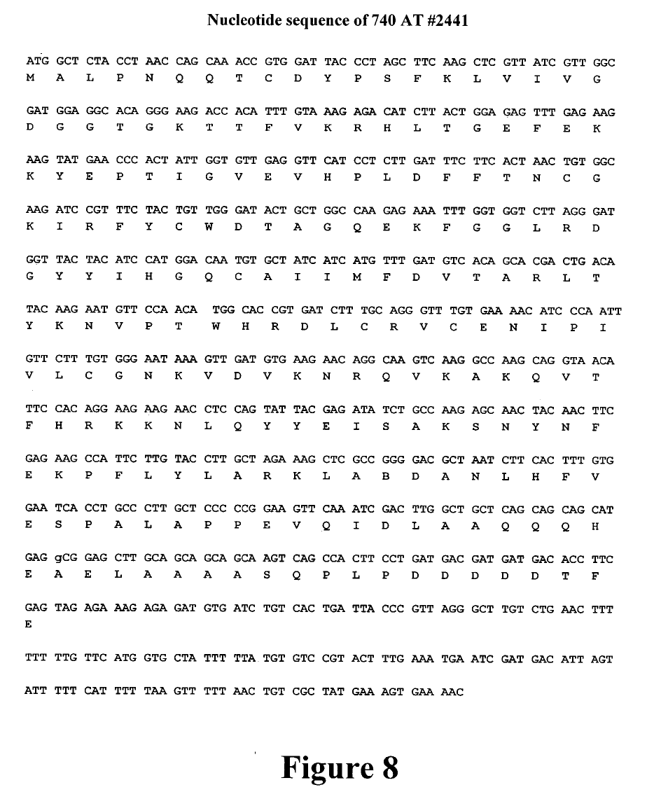

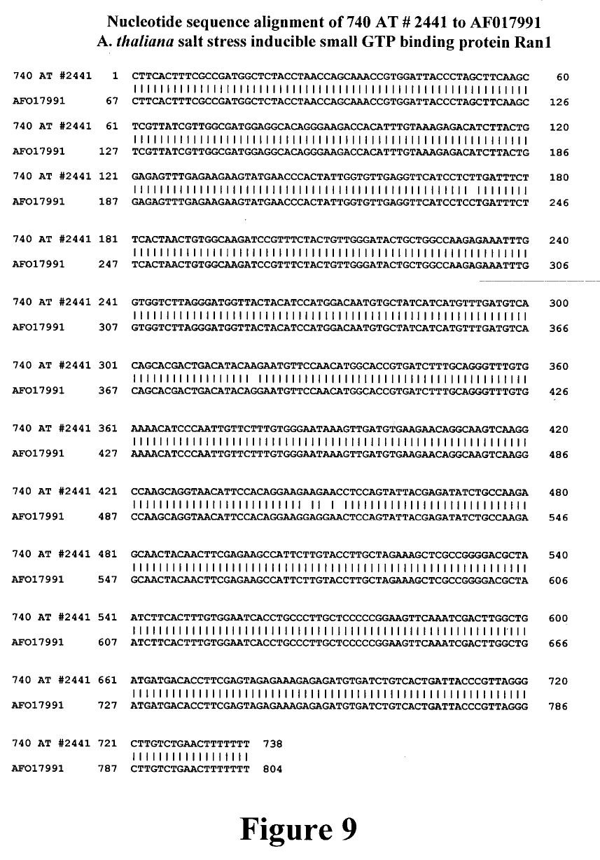

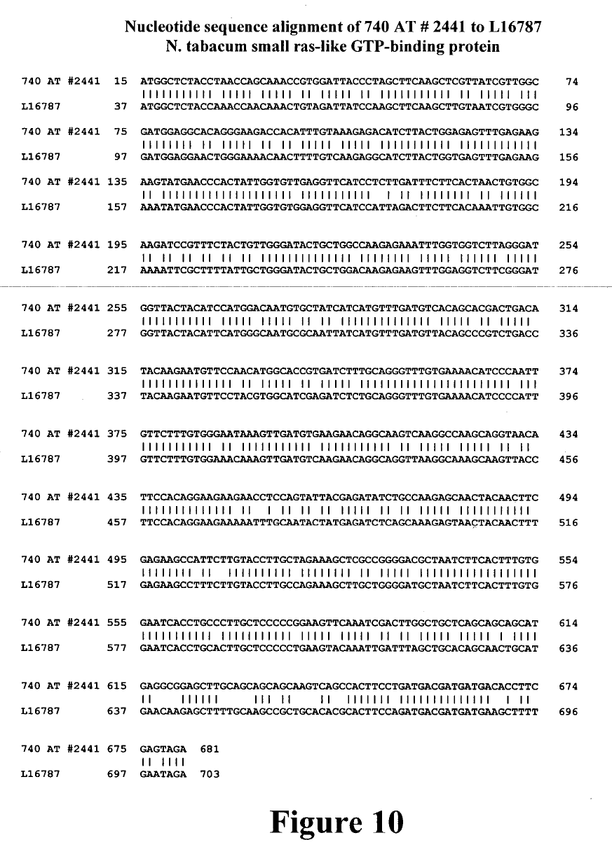

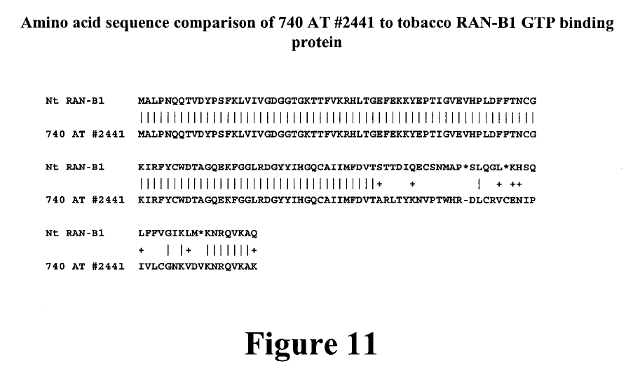

A 841 bp NotI fragment of 740 AT #2441 (FIG. 8, SEQ ID NOs:29 and 30) containing the Ran GTP binding protein cDNA was characterized. The nucleotide sequencing of 740 AT #2441 was carried out by dideoxy termination using double stranded templates (Sanger et al. 1977). Nucleotide sequence analysis and amino acid sequence comparisons were performed using DNA Strider, PCGENE and NCBI Blast programs. 740 AT #2441 contained an open reading frame (ORF) in the positive orientation that encodes a protein of 221 amino acids with an apparent molecular weight of 25,058 Da. The mass of the protein was calculated using the Voyager program (Perceptive Biosystems). FIG. 9 shows the nucleotide sequence alignment of 740AT #2441 to AF017991, a A. thaliana salt stress inducible small GTP binding protein Ran1. FIG. 10 shows the nucleotide alignment of 740 AT #2441 to L16787, a N. tabacum small ras-like GTP binding protein (SEQ ID NO: 32). FIG. 11 shows the amino acid comparison of 740 AT #2441 (SEQ ID NO: 33) to tobacco Ran-B 1 GTP binding protein (SEQ ID NO: 34). The A. thaliana cDNA exhibits a high degree of homology (99% to 82%) to A. thaliana, tomato (L. esculentum), tobacco (N. tabacum), L. japonicus and bean (V. faba) GTP binding proteins cDNAs (TABLE 1). The nucleotide sequence from 740 AT #2441 encodes a protein that has strong similarity (100% to 95%) to A. thaliana, tomato, tobacco, and bean GTP binding proteins (TABLE 2).

MALDI-TOF analysis of N. benthamiana transfected with 740 AT #2441

10 days after inoculation, the apical meristem, leaves, and stems from N. benthamiana transfected with 740 AT #2441, were frozen in liquid nitrogen. The soluble proteins were extracted in grinding buffer (100 mM Tris, pH 7.5, 2 mM EDTA, 1 mM PMSF, 10 mM BME) using a mortar and pestle. The homogenate was filtered through four layers of cheesecloth and spun at 10,000×g for 15 min. The supernatant was decanted and spun at 100,000×g for 1 hr. A 500 μl aliquot of the supernant was mixed with 500 μl 20% TCA (acetone/0.07% BME) and stored at 4° C. overnight. The supernant was analyzed by MALDI-TOF. (Karas et al., Anal. Chem., 60:2299-2301 (1988)). The results showed that the soluble proteins contained a newly expressed protein of molecular weight 25,058.

Isolation of an Arabidopsis thaliana GTP Binding Protein Genomic Clone

SA genomic clone encoding A. thaliana GTP binding proteins can be isolated by probing filters containing A. thaliana BAC clones using a ³²P-labelled 740 AT #2441 NotI insert. Other members of the A. thaliana ARF multigene family have been identified using programs of the University of Wisconsin Genetic Computer Group.

TABLE 1
740 AT #2441 Nucleotide sequence comparison

| Clone | Score | pValue | Identities | Positives |
|---|---|---|---|---|
| A. thaliana AF017991 | 3645 (1007 .2 bits) | 0.00E+00 | 773/738 (99%) | 733/738 (99%) |
| A. thalianai ATU75601 | 3645 (1007 .2 bits) | 0.00E+00 | 733/738 (99%) | 773/778 (99%) |
| A. thalianai X97381 | 3618 (999.7 bits) | 0.00E+00 | 730/738 (99%) | 733/738 (98%) |
| L. esculentum L28714 | 2341 (646.9 bits) | 1.50E−189 | 561/677 (82%) | 561/677 (82%) |
| N. tabacum L16787 | 2336 (645.5 bits) | 3.90E−189 | 556/667 (83%) | 556/667 (83%) |
| L. esculentum L28713 | 2313 (639.1 bits) | 3.00E−187 | 557/675 (82%) | 557/675 (82%) |
| L. esculentum L28715 | 2336 (645.5 bits) | 4.10E−189 | 560/676 (82%) | 560/676 (82%) |
| V. faba Z24678 | 2325 (642.4 bits) | 3.30E−188 | 557/672 (82%) | 557/672 (82%) |
| N. tabacum L16767 | 2272 (627.8 bits) | 7.70E−184 | 548/665 (82% | 548/665 (82%) |
| L. japonicus Z73960 | 2194 (606.2 bits) | 3.00E−177 | 526/635 (82%) | 526/635 (82%) |
| L. japonicus Z73959 | 2187 (604.3 bits) | 9.70E−177 | 531/648 (81%) | 531/648 (81%) |

TABLE 2
740 AT #2441 Amino acid sequence comparison

| Clone | Score | pValue | Identities | Positives |
|---|---|---|---|---|
| A. thaliana SP_PL: O04664 | 1192 (554.1 bits) | 1.50E−162 | 221/221 | 221/221 |
| | | | (100%) | (100%) |
| A. thaliana SP_PL: O04148 | 1172 (544.8 bits) | 9.60E−160 | 217/221 (98%) | 220/221 (99%) |
| A. thaliana SP_PL: O22495 | 1172 (544.8 bits) | 9.60E−160 | 217/221 (98%) | 219/221 (99%) |
| N. tabacum SW: RANA_TOBAC P41918 | 1169 (543.4 bits) | 2.50E−159 | 216/221 (97%) | 218/221 (98%) |
| V. faba SW: RAN_VICFA P38548 | 1155 (536.9 bits) | 2.30E−157 | 212/221 (95%) | 216/221 (97%) |
| A. thaliana SW: RAN2_ARATH P41917 | 1150 (534.6 bits) | 1.10E−156 | 211/221 (95%) | 217/221 (98%) |
| L. esculentum SW: RAN2_LYCES P38547 | 1148 (533.7 bits) | 2.20E−156 | 212/221 (95%) | 214/221 (96%) |
| N. tabacum SW: RANB_TOBAC P41919 | 1145 (532.3 bits) | 5.70E−156 | 212/221 (95%) | 214/221 (96%) |
| L. esculentum SW: RAN1_LYCES P38546 | 1143 (531.3 bits) | 1.10E−155 | 211/221 (95%) | 213/221 (96%) |
| A. thaliana SW: RAN1_ARATH P41916 | 1141 (530.4 bits) | 2.10E−155 | 210/221 (95%) | 215/221 (97%) |
| L. japonicus SW: RANB_LOTJA P54766 | 1111 (516.5 bits) | 3.50E−151 | 205/209 (98%) | 207/209 (99%) |
| L. japonicus SW: RANA_LOTJA P 54765 | 1106 (514.1 bits) | 1.70E−150 | 204/209 (97%) | 206/209 (98%) |

### Example 12

Construction of a Cytoplasmic Inhibition Vector Containing A. thaliana Ribulose Bisphosphate Carboxylase Small Subunit (Rubisco) Nucleotide Sequence.

An Arabidopsis thaliana CD4-13 cDNA library was digested with NotI. DNA fragments between 500 and 1000 bp were isolated by trough elution and subcloned into the NotI site of pBS740. E. coli C600 competent cells were transformed with the pBS740 AT library and colonies containing Arabidopsis cDNA sequences were selected on LB Amp 50 μg/ml.

Isolation of a Gene Encoding Ribulose Bisphosphate Carboxylase (Rubisco) Small Subunit.

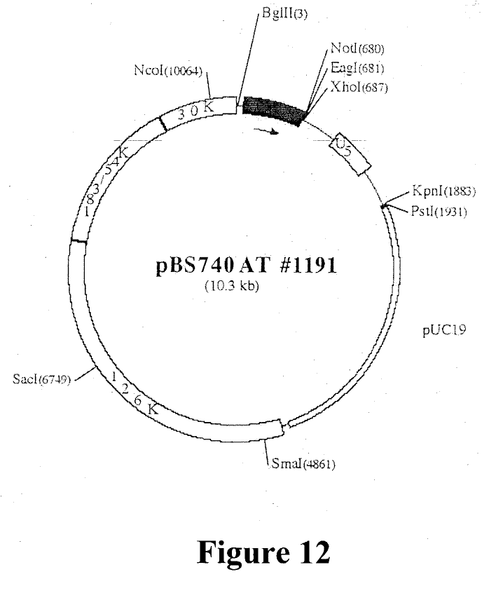

One to two weeks after inoculation, transfected Nicotiana benthamiana plants were visually monitored for changes in growth rates, morphology, and color. Plants transfected with 740 AT #1191 (FIG. 12) developed etched yellow concentric rings around the systemically infected veins. Plasmid 740 AT #1191 contains the TMV-U1 126-, 193-, and 30-kDa ORFs, the TMV-U5 coat protein gene (U5 cp), the T7 promoter, an Arabidopsis thaliana CD4-13 NotI fragment, and part of the pUC19 plasmid. The TMV-U1 subgenomic promoter located within the minus strand of the 30-kDa ORF controls the syntehsis of the CD4-13 subgenomic RNA.

DNA Sequencing and Computer Analysis.

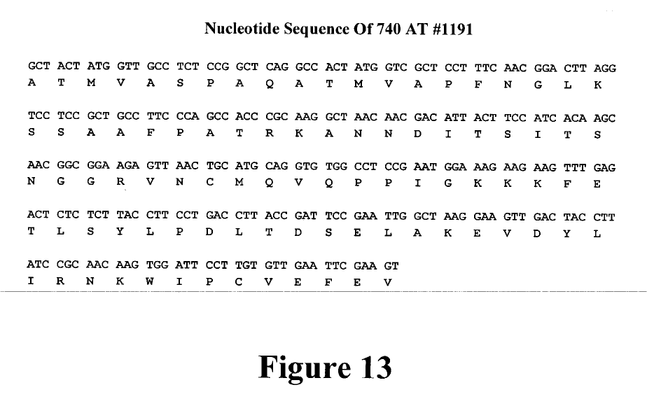

The NotI fragment of 740 AT #1191 was characterized: nucleotide sequence analysis and amino acid sequence comparisons were performed using DNA Strider, PCGENE and NCBI Blast programs. 740 AT #1191 contained a partial open reading frame (ORF) of Rubisco in the positive orientation (FIG. 13, SEQ ID NO. 35), the start codon of which is deleted from the wild type. 740 AT #1191 encodes a partial A. thaliana ribulose bisphosphate carboxylase (FIG. 13, SEQ ID NO: 36), which is a highly expressed protein in plants. The expression of Rubisco was inhibited in the transfected N. benthamina because 740 AT #1191 contained only a partial Ribisco small unit cDNA, without a start codon.

### Example 13

Construction of a Cytoplasmic Inhibition Vector Containing A. thaliana HAT7 Homeobox-Leucine Zipper Nucleotide Sequence.

An Arabidopsis thaliana CD4-13 cDNA library was digested with NotI. DNA fragments between 500 and 1000 bp were isolated by trough elution and subcloned into the NotI site of pBS740. E. coli C600 competent cells were transformed with the pBS740 AT library and colonies containing Arabidopsis cDNA sequences were selected on LB Amp 50 μg/ml.

Isolation of a Gene Encoding HAT7 Homeobox-Leucine Zipper.

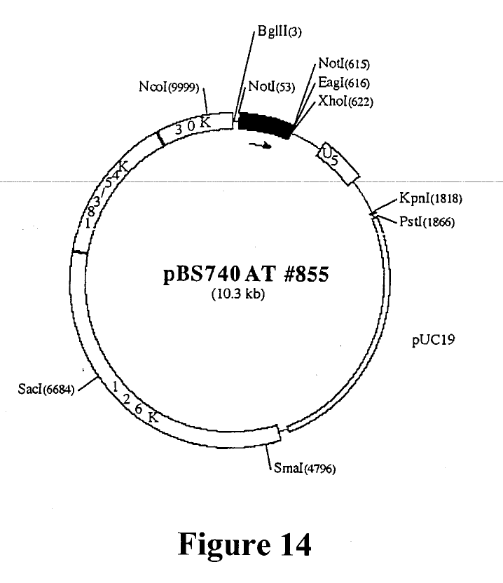

One to two weeks after inoculation, transfected Nicotiana benthamiana plants were visually monitored for changes in growth rates, morphology, and color. Plants transfected with 740 AT #855 (FIG. 14) were moderately stunted. Plasmid 740 AT #855 contains the TMV-UI 126-, 193-, and 30-kDa ORFs, the TMV-U5 coat protein gene (U5 cp), the T7 promoter, an Arabidopsis thaliana CD4-13 NotI fragment, and part of the pUC19 plasmid. The TMV-U1 subgenomic promoter located within the minus strand of the 30-kDa ORF controls the syntehsis of the CD4-13 subgenomic RNA.

DNA Sequencing and Computer Analysis.

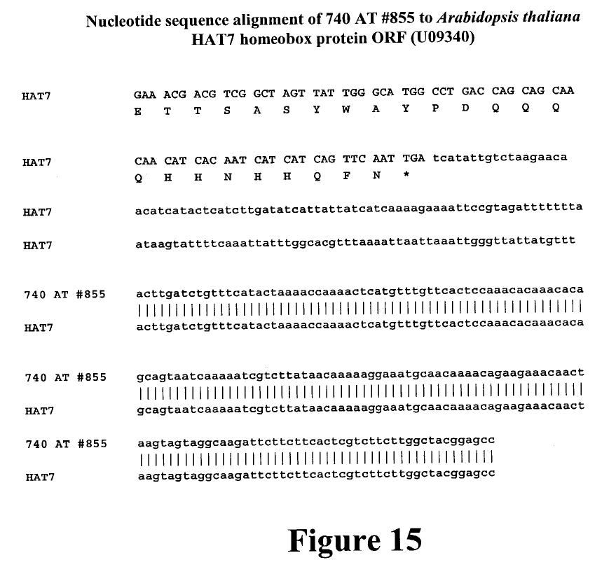

The NotI fragment of 740 AT #855 was characterized: nucleotide sequence analysis and amino acid sequence comparisons were performed using DNA Strider, PCGENE and NCBI Blast programs 740 AT #855 contained A. thaliana HAT 7 homeobox-luecine zipper cDNA sequence. The nucleotide sequence alignment of 740 AT #855 and Arabidopsis thaliana HAT7 homeobox protein ORF (U09340) was compared. FIG. 15 shows the nucleotide sequences of 740 #855 (SEQ ID NO: 37) and A. thaliana HAT7 homeobox protein ORF (SEQ ID NO: 38), and the amino acid sequence of A. thaliana HAT7 homeobox protein ORFs (SEQ ID NO: 39). The result show that 740 AT #855 contains a 3′-untranslated region (UTR) of the A. thaliana HAT7 homeobox protein ORF in a positive orientation, thus inhibited the expression of HAT 7 homeobox protein in the transfected N. benthamiana.

### Example 14

Construction of a Nicotiana benthamiana cDNA Library.

Vegetative N. benthamiana plants were harvested 3.3 weeks after sowing and sliced up into three groups of tissue: leaves, stems and roots. Each group of tissue was flash frozen in liquid nitrogen and total RNA was isolated from each group separately using the following hot borate method (Larry Smart and Thea Wilkins, 1995). Frozen tissue was ground to a fine powder with a pre-chilled mortar and pestle, and then further homogenized in pre-chilled glass tissue grinder. Immediately thereafter, 2.5 ml/g tissue hot (˜82° C.) XT Buffer (0.2 M borate decahydrate, 30 mM EGTA, 1% (w/v) SDS. Adjusted pH to 9.0 with 5 N NaOH, treated with 0.1% DEPC and autoclaved. Before use, added 1% deoxycholate (sodium salt), 10 mM dithiothreitol, 15 Nonidet P-40 (NP-40) and 2% (w/v) polyvinylpyrrilidone, MW 40,000 (PVP-40)) was added to the ground tissue. The tissue was homogenized 1-2 minutes and quickly decanted to a pre-chilled Oak Ridge centrifuge tube containing 105 μl of 20 mg/ml proteinase K in DEPC treated water. The tissue grinder was rinsed with an additional 1 ml hot XT Buffer per g tissue, which was then added to rest of the homogenate. The homogenate was incubated at 42° C. at 100 rpm for 1.5 h. 2 M KCl was added to the homogenate to a final concentration of 160 mM, and the mixture was incubated on ice for 1 h to precipitate out proteins. The homogenate was centrifuged at 12,000×g for 20 min at 4° C., and the supernatant was filtered through sterile miracloth into a clean 50 ml Oak Ridge centrifuge tube. 8 M LiCl was added to a final concentration of 2 M LiCl and incubated on ice overnight. Precipitated RNA was collected by centrifugation at 12,000×g for 20 min at 4° C. The pellet was washed three times in 3-5 ml 4° C. 2 M LiCl. Each time the pellet was resuspended with a glass rod and then spun at 12,000×g for 20 min at 4° C. The RNA pellet was suspended in 2 ml 10 mM Tris-HCl (pH 7.5), and purified from insoluble cellular components by spinning at 12,000×g for 20 min at 4° C. The RNA containing supernatant was transferred to a 15 ml Corex tube and precipitated overnight at −20° C. with 2.5 volumes of 100% ethanol. The RNA was pelleted by centrifugation at 9,800×g for 30 min at 4° C. The RNA pellet was washed in 1-2 ml cold 70° C. ethanol and centrifuged at 9,800×g for 5 min at 4° C. Residual ethanol was removed from the RNA pellet under vacuum, and the RNA was resuspended in 200 μl DEPC treated dd-water and transferred to a 1.5 ml microfuge tube. The Corex tube was rinsed in 100 μl DEPC-treated dd-water, which was then added to the rest of the RNA. The RNA was then precipitated with 1/10 volume of 3 M sodium acetate, pH 6.0 and 2.5 volumes of cold 100% ethanol at −20° C. for 1-2 h. The tube was centrifuged for 20 min at 16,000×g, and the RNA pellet washed with cold 70% ethanol, and centrifuged for 5 min at 16,000×g. After drying the pellet under vacuum, the RNA was resuspended in DEPC-treated water. This is the total RNA.

Messenger RNA was purified from total RNA using an Poly(A)Pure kit (Ambion, Austin Tex.), following the manufacturer's instructions. A reverse transcription reaction was used to synthesize cDNA from the mRNA template, using either the Stratagene (La Jolla, Calif.) or Gibco BRL (Gaithersburg, Md.) cDNA cloning kits. For the Stratagene library, the cDNAs were subcloned into bacteriophage at EcoR1/XhoI site by ligating the arms using the Gigapack III Gold kit (Stratagene, La Jolla, Calif.), following the manufacturer's instructions. For the Gibco BRL library, the cDNAs were subcloned into a tobamoviral vector that contained a fusion of TMV-UL and TMV-U5 at the NotI/XhoI sites.

### Example 15

Rapid Isolation of cDNAs Encoding GTP Binding Proteins from Rice, Barley, Corn, Soybean, and Other Plants

Libraries containing full-length cDNAs from rice, barley, corn, soybean and other important crops are obtained from public and private sources or can be prepared from plant mRNAs. The cDNAs are inserted in viral vectors or in small subcloning vectors such as pBluescript (Strategene), pUC18, M13, or pBR322. Transformed bacteria (E. coli) are then plated on large petri plates or bioassay plates containing the appropriate media and antibiotic. Individual clones are selected using a robotic colony picker and arrayed into 96 well microtiter plates. The cultures are incubated at 37° C. until the transformed cells reach log phase. Aliquots are removed to prepare glycerol stocks for long term storage at −80° C. The remainder of the culture is used to inoculate an additional 96 well microtiter plate containing selective media and grown overnight. DNAs are isolated from the cultures and stored at −20° C. Using a robotic unit such as the Qbot (Genetix), the E. coli transformants or DNAs are rearrayed at high density on nylon filters or glass slides. Full-length cDNAs encoding GTP binding proteins from rice, barley, corn, soybean and other important crops are isolated by screening the various filters of slides by hybridization using a ³²P-labeled or fluorescent-labeled 740 AT #2441 Not I insert.

### Example 16

Rapid Isolation of Genomic Clones Encoding GTP Binding Proteins Factor from Rice, Barley, Corn, Soybean, and Other Plant

Genomic libraries containing sequences from rice, barley, corn, soybean and other important crops are obtained from public and private sources, or are prepared from plant genomic DNAs. BAC clones containing entire plant genomes have been constructed and organized in minimal overlappping order. Individual BACs are sheared to 500-1000 bp fragments and directly cloned into viral vectors. Approximate 200-500 clones that completely cover an entire BAC will form a BAC viral vector sublibrary. These libraries can be stored as bacterial glycerol stocks at −80C and as DNA at −20C. Genomic clones are identified by first probing filters of BACs with a ³²P-labeled or fluorescent-labeled 740 AT #2441 Not I insert. BACs that hybridize to the probe are selected and their corresponding BAC viral vector is used to produce infectious RNA. Plants that are transfected with the BAC sublibrary are screened for change of function (for example, stunted plants). The inserts from these clones or their corresponding plasmid DNAs are characterized by dideoxy sequencing. This provides a rapid method to obtain the genomic sequence for the plant GTP binding proteins.

### Example 17

Novel Requirements for Production of Infectious Viral Vector in vitro Derived RNA Transcripts.

This example demonstrates the production of highly infectious viral vector transcripts containing 5′ nucleotides with reference to the virus vector.

Construction of a library of subgenomic cDNA clones of TMV and BMV has been described in Dawson et al., Proc. Natl. Acad. Sci. USA 83:1832-1836 (1986) and Ahlquist et al., Proc. Natl. Acad. Sci. USA 81:7066-7070 (1984). Nucleotides were added between the transcriptional start site of the promoter for in vitro transcription, in this case T7, and the start of the cDNA of TMV in order to maximize transcription product yield and possibly obviate the need to cap virus transcripts to insure infectivity. The relevant sequence is the T7 promoter . . . TATAG\hat{ }ATATTTT (SEQ ID NO: 40). . . . where the \hat{ } indicates the base preceding is the start site for transcription and the bold letter is the first base of the TMV cDNA. Three approaches were taken: 1) addition of G, GG or GGG between the start site of transcription and the TMV cDNA (. . . TATAGGTATTT, SEQ ID NO: 41, . . . and associated sequences); 2) addition of G and a random base (GN or N2) or a G and two random bases (GNN or N3) between the start site of transcription and the TMV cDNA (. . . TATAGNTATTT, SEQ ID No:42, . . . and associated sequences), and the addition of a GT and a single random base between the start site of transcription and the TMV cDNA (. . . TATAGTNGTATTT . . . and associated sequences). The use of random bases was based on the hypothesis that a particular base may be best suited for an additional nucleotide attached to the cDNA, since it will be complementary to the normal nontemplated base incorporated at the 3′-end of the TMV (−) strand RNA. This allows for more ready mis-initiation and restoration of wild type sequence. The GTN would allow the mimicking of two potential sites for initiation, the added and the native sequence, and facilitate more ready mis-initiation of transcription in vivo to restore the native TMV cDNA sequence. Approaches included cloning GFP expressing TMV vector sequences into vectors containing extra G, GG or GGG bases using standard molecular biology techniques. Likewise, full length PCR of TMV expression clone 1056 was done to add N2, N3 and GTN bases between the T7 promoter and the TMV cDNA. Subsequently, these PCR products were cloned into pUC based vectors. Capped and uncapped transcripts were made in vitro and inoculated to tobacco protoplasts or Nicotiana benthamiana plants, wild type and 30k expressing transgenics. The results are that an extra

| | |
|---|---|
| G, . . . TATAGGTATTTT, . . . or a | SEQ ID NO:43 |
| GTC, . . . TATAGTCGTATTTT, . . ., | SEQ ID NO:44 |

were found to be well tolerated as additional 5′ nucleotides on the 5′ of TMV vector RNA transcripts and were quite infectious on both plant types and protoplasts as capped or non-capped transcripts. Other sequences may be screened to find other options. Clearly, infectious transcripts may be derived with extra 5′ nucleotides.

Other derivatives based on the putative mechanistic function of the GTN strategy that yielded the GTC functional vector are to use multiple GTN motifs preceding the 5′ most nt of the virus cDNA or the duplication of larger regions of the 5′-end of the TMV genome. For example:

| | |
|---|---|
| TATA\hat{ }GTNGTNGTATT, . . . or, | SEQ ID NO:45 |
| TATA\hat{ }GTNGTNGTNGTNGTATT, . . . or | SEQ ID NO:46 |
| TATA\hat{ }GTATTTGTATTT, . . . | SEQ ID NO:47 |

In this manner the replication mediated repair mechanism may be potentiated by the use of multiple recognition sequences at the 5′-end of transcribed RNA. The replicated progeny may exhibit the results of reversion events that would yield the wild type virus 5′ virus sequence, but may include portions or entire sets of introduced additional base sequences. This strategy can be applied to a range of RNA viruses or RNA viral vectors of various genetic arrangements derived from wild type virus genome. This would require the use of sequences particular to that of the virus used as a vector.

### Example 18

Infectivity of Uncapped Transcripts.

Two TMV-based virus expression vectors were initially used in these studies pBTI 1056 which contains the T7 promoter followed directly by the virus cDNA sequence (. . . TATAGTATT . . . ), and pBTI SBS60-29 which contains the T7 promoter (underlined) followed by an extra guanine residue then the virus cDNA sequence (. . . TATAGGTATT . . . ). Both expression vectors express the cycle 3 shuffled green fluorescent protein (GFPc3) in localized infection sites and systemically infected tissue of infected plants. Transcriptions of each plasmid were carried out in the absence of cap analogue (uncapped) or in the presence of 8-fold greater concentration of RNA cap analogue than rGTP (capped). Transcriptions were mixed with abrasive and inoculated on expanded older leaves of a wild type Nicotiana benthamiana (Nb) plant and a Nb plant expressing a TMV U1 30k movement protein transgene (Nb 30K). Four days post inoculation (dpi), long wave UV light was used to judge the number of infection sites on the inoculated leaves of the plants. Systemic, noninoculated tissues, were monitored from 4 dpi on for appearance of systemic infection indicating vascular movement of the inoculated virus. Table 3 shows data from one representative experiment.

TABLE 3

| | |
|---|---|
| Local infection sites | Systemic Infection |

| Construct | Nb | Nb 30K | Nb | Nb 30K |
|---|---|---|---|---|
| pBTI1056 | | | | |
| Capped | 5 | 6 | yes | yes |
| Uncapped | 0 | 5 | no | yes |
| PBTI SBS60-29 | | | | |
| Capped | 6 | 6 | yes | yes |
| Uncapped | 1 | 5 | yes | yes |

Nicotiana tabacum protoplasts were infected with either capped or uncapped transcriptions (as described above) of pBTI SBS60 which contains the T7 promoter followed directly by the virus cDNA sequence (TATAGTATT . . . ). This expression vector also expresses the GFPc3 gene in infected cells and tissues. Nicotiana tabacum protoplasts were transfected with 1 mcl of each transcriptions. Approximately 36 hours post infection transfected protoplasts were viewed under UV illumination and cells showing GFPc3 expression. Approximately 80% cells transfected with the capped PBTI SBS60 transcripts showed GFP expression while 5% of cells transfected with uncapped transcripts showed GFP expression. These experiments were repeated with higher amounts of uncapped inoculum. In this case a higher proportion of cells, >30% were found to be infected at this time with uncapped transcripts, where >90% of cells infected with greater amounts of capped transcripts were scored infected. These results indicate that, contrary to the practiced art in scientific literature and in issued patents (Ahlquist et al., U.S. Pat. No. 5,466,788), uncapped transcripts for virus expression vectors are infective on both plants and in plant cells, however with much lower specific infectivity. Therefore, capping is not a prerequisite for establishing an infection of a virus expression vector in plants; capping just increases the efficiency of infection. This reduced efficiency can be overcome, to some extent, by providing excess in vitro transcription product in an infection reaction for plants or plant cells.

The expression of the 30K movement protein of TMV in transgenic plants also has the unexpected effect of equalizing the relative specific infectivity of uncapped verses capped transcripts. The mechanism behind this effect is not fully understood, but could arise from the RNA binding activity of the movement protein stabilizing the uncapped transcript in infected cells from prereplication cytosolic degradation.

Extra guanine residues located between the T7 promoter and the first base of a virus cDNA lead to increased amount of RNA transcript as predicted by previous work with phage polymerases. These polymerases tend to initiate more efficiently at . . . TATAGG or . . . TATAGGG than . . . TATAG. This has an indirect effect on the relative infectivity of uncapped transcripts in that greater amounts are synthesized per reaction resulting in enhanced infectivity.

Data Concerning Cap Dependent Transcription of pBTI1056 GTN#28.

TMV-based virus expression vector pBTI 1056 GTN#28 which contains the T7 promoter (underlined) followed GTC bases (bold) then the virus cDNA sequence (. . . TATAGTCGTATT, SEQ ID NO: 48, . . .). This expression vector expresses the cycle 3 shuffled green fluorescent protein (GFPc3) in localized infection sites and systemically infected tissue of infected plants. This vector was transcribed in vitro in the presence (capped) and absence (uncapped) of cap analogue. Transcriptions were mixed with abrasive and inoculated on expanded older leaves of a wild type Nicotiana benthamiana (Nb) plant and a Nb plant expressing a TMV U1 30k movement protein transgene (Nb 30K). Four days post inoculation (dpi) long wave UV light was used to judge the number of infection sites on the inoculated leaves of the plants. Systemic, non-inoculated tissues, were monitored from 4 dpi on for appearance of systemic infection indicating vascular movement of the inoculated virus. Table 4 shows data from two representative experiments at 11 dpi.

TABLE 4

| | |
|---|---|
| Local infection sites | Systemic Infection |

| Construct | Nb | Nb 30K | Nb | Nb 30K |
|---|---|---|---|---|
| Experiment 1 | | | | |
| pBTI1056 GTN#28 | | | | |
| Capped | 18 | 25 | yes | yes |
| Uncapped | 2 | 4 | yes | yes |
| Experiment 2 | | | | |
| pBTI1056 GTN#28 | | | | |
| Capped | 8 | 12 | yes | yes |
| Uncapped | 3 | 7 | yes | yes |

These data further support the claims concerning the utility of uncapped transcripts to initiate infections by plant virus expression vectors and further demonstrates that the introduction of extra, non-viral nucleotides at the 5′-end of in vitro transcripts does not preclude infectivity of uncapped transcripts.

### Example 19

Methods for Inhibiting Endogenous Proteolytic Activity in Plants in vivo.

Elicitor recognition and the response cascades occurring in plants form an essential link between the environmental stress and plant survival responses. Many products are induced following induction by environmental stimuli or pathogen infection, which include, but are not limited to, proteases, protease inhibitors, alkaloids and other metabolites. Glazebrook, et al., Annu. Rev. Gen. 31:547-569 (1997); Grahm, et al., J. Biol. Chem. 260:6555-6560 (1985); and Ryan, et al., Ann. Rev. Cell Dev. Biol. 14:1-17 (1998), all incorporated herein by reference. The components of the recognition and response pathways are poorly understood, yet have tremendous practical value for input traits in genetically improved crops. Traditional methods of mutagenesis or biochemistry are leading to slow and incremental advances in our understanding. However, if these pathways are to be elucidated, understood and exploited, more rapid discovery methods must be brought to bear on the problem. Virus expression vectors capable of either overexpressing gene products or suppressing the expression of particular endogenous host genes provide a unique tool to discover the nature of the genes whose products contribute to the response pathways.

This example describes methods for inhibiting endogenous plant proteases which interfere with the expression and purification of recombinant proteins in plants. In particular, this example shows methods for inhibiting proteolytic activity in planta which is responsible for the degradation of a viral vector-expressed recombinant protein. These methods are also applicable to the protection of recombinant proteins expressed via a stable transformation system or endogenous plant proteins. Viral vectors have been constructed to include an N-terminal signal peptide sequence. This sequence directs the recombinant protein through the secretory pathway to the cell surface and ultimately accumulating in the plant intercellular fluid (IF) (Kermode, Critical Reviews in Plant Sciences 15(4):285-423 (1996), incorporated herein by reference). In some instances, the target protein was cleaved aberrantly in vivo. Three examples include a mammalian growth hormone and single chain antibody and an avian interferon. In vivo residence time in the IF led to the accumulation of the cleavage product(s) as detected by immunoblotting. Cleavage was either complete in vivo or continued in vitro following IF extraction (Co-pending U.S. patent application Ser. No. 09/037,751, incorporated herein by reference). Quantitation of western blots using UVP Gelbase/Gelblot-Pro software revealed as much as 40-50% of the expressed protein was cleaved.

We designed in vitro experiments to inhibit the plant proteolytic activity. When we added protease inhibitors to an isolated IF fraction in vitro, we were able to inhibit further degradation of our recombinant protein. In addition, when we treated an IF fraction from an unrelated virally infected plant with protease inhibitors and incubated that with a known susceptible protein, we completely inhibited the protease and protected the protein from degradation.

Following the observation that the cleavage was occurring in vivo by a plant protease that could be inhibited by proteinase inhibitors, we designed experiments to inhibit this activity in planta. Three possible methods to inhibit the protease are as follows:

1. Recombinant Expression of a Proteinase Inhibitor:

The activity of the plant protease may be inhibited by the recombinant expression of a plant proteinase inhibitor secreted to the IF based on the following results:

(1) We cloned a tomato proteinase inhibitor gene (Wingate, et al., J. Biol. Chem. 264:17734-17738 (1989), incorporated herein by reference) into our viral vector. We verified that the expression of the recombinant inhibitor protein was in the IF fraction by western detection. Virally-expressed proteinase inhibitor protected our recombinant (E. coli-derived) mammalian growth hormone protein standard that was known to be susceptible to the plant protease in an in vitro assay;

(2) Virally-expressed proteinase inhibitor specifically inhibited an IF-localized protease in vivo as per detection on Zymogram gelatin Tris-glycine gels; and

(3) Co-inoculation of the virus vector proteinase inhibitor construct and the viral vector mammalian growth hormone construct resulted in the expression of both proteins in systemic leaves and partial protection of the growth hormone in the IF.

Another possible approach is to combine transgenic plants and virally-expressed proteins. One could either inoculate the virus vector proteinase inhibitor construct on transgenic plants expressing a target protein or make a proteinase inhibitor transgenic plant and inoculate with a viral vector construct expressing the target sequence.

2. Induction of Endogenous Proteinase Inhibitors:

One could also induce the endogenous production of plant proteinase inhibitors using an elicitor. For example, jasmonic acid (JA) is produced as part of a general plant defense mechanism and is known to induce specific proteinase inhibitors (Lightner et al., J Mol Gen Genet. 241:595-601 (1993), incorporated herein by reference). Exogenous application of JA as been used to induce a plant defense response in Nicotiana attenuata to against herbivore attack (Baldwin, PNAS, 95(14):8113-8118 (1998), incorporated herein by reference). To protect against specific endogenous proteolysis of a recombinant protein, one could treat the plant material with JA to induce the synthesis of the proteinase inhibitor and then inoculate with a viral vector construct expressing the target sequence.

The desired phenotype in host plants used for gene discovery program using virus expression vectors is reduced proteolytic activities in the cytosol, secretory pathway or apoplast so to increase the half-life of virally produced proteins. This will allow virally expressed proteins to exert their influence on plant biochemistry, development and growth optimally. Rapid or premature degradation may reduce the amount of the expressed protein below the necessary threshold to exert a measurable effect. Transgenic expression of protease inhibitors, such as those induced by the systemin pathway (Ryan, et al., Ann. Rev. Cell Dev. Biol. 14:1-17 (1998)), will provide a continuous source of inhibitor to slow particular degradation processes. Conversely, as outlined in the example above, treating virus vector infected plants with JA will induce the response pathways and result in the expression of various inhibitors in infected/treated plants. In both ways, by specific protease inhibitor expression or by induction of response cascade, the half-lives of many proteins, whose presence is requisite for detecting the novel functions of gene products, are increased.

### Example 20

Purification and Biochemical Characterization of N. benthamianai Subtilisin-like Serine Protease

Inhibiting plant proteases during a genomics discovery effort allows for a broader spectrum of genes expressed in a host plant to be scored. This occurs in two ways. First the proteolytic degradation of recombinant proteins is inhibited, allowing the research to see the effects of the protein on the plant. Second, the proteolytic degradation of endogenous host plant proteins is inhibited, allowing recombinant proteins to interact with a broader spectrum of endogenous proteins of the plant host.

Plant Material

N. benthamiana plants were grown in a controlled environment with 27 C day and 23 C night temperatures, a 12 h photoperiod, and 86% relative humidity. Plants used for protease purification and characterization were inoculated three weeks post sow date with in vitro infectious transcripts of a plant viral vector encoding an hGH gene sequence in-frame with a tobacco extensin signal peptide as previously described for other tobamovirus expression studies (20,21).

Plant IF Protease Inhibitor Studies

Eight to ten days post inoculation (dpi), infected plant material was harvested and used to isolate the plant interstitial fluid (IF) as previously described. Briefly, infected leaf and stem material was vacuum infiltrated with infiltration buffer (100 mM Tris-HCl, pH 7.0, 2 mM EDTA and 0.1% beta-mercaptoethanol) and secreted proteins were recovered by centrifugation. A 5 μl aliquot of infected plant material IF was flash/frozen with or without the addition of chymostatin (100 μM), kept frozen or lyophilized and kept at −20 C for 2 days. Samples were thawed or reconstituted and stability analyzed following 3 days at 4 C. An aliquot of infected plant IF was also treated with potato PI-I inhibitor (gift of Dr. Clarence Ryan, Washington State University) or made to 1.0 mM PMSF (Sigma). The stability of hGH (human growth hormone) in the inhibitor-treated IF fractions was monitored by anti-hGH western detection.

Electrophoresis and Immunoblotting

SDS-PAGE was performed on precast gels (Novex, San Diego) in the buffer system of Laemmli with a Xcell II Mini-Cell apparatus (Novex). Proteins were electrophoretically transferred (1 hr, 100 v, 4 C) to nitrocellulose membrane (0.45 um, Schleicher & Schuell) or PVDF membrane in standard tris-glycine buffer with 20% methanol.

Proteolytic Degradation of hGH

Stability of the plant virally-expressed hGH protein was monitored using western blot detection. Immunoblots were blocked with 5% nonfat dry milk in TBST for 1 hr, then probed with anti-hGH antibody (Biodesign International). Crossreacting proteins and peptides were detected using enhanced chemiluminescence (ECL, Amersham) or goat anti-rabbit IgGT conjugate to alkaline phosphate (Sigma) as directed by the manufacturers. Plant viral vectors have been used successfully to express high levels of recombinant proteins in plants. It has been a particularly useful system for the expression and subsequent purification of secreted proteins. Unfortunately, efforts in this study to produce a secreted human growth hormone using a tobamovirus vector in N. benthamiana, resulted in the accumulation of peptide fragments that crossreacted with the hGH antibody and very little uncleaved protein. The molecular weight of intermediate hGH degradation product detected in the plant interstitial fluid (IF) was approximately 16 kD by the Western blot analysis. These sizes matched well with the reported size of specific hGH peptide fragments that were detected in vivo and generated by in vitro studies, suggesting a similar processing may be occurring in planta as in mammalian systems.

In an effort to identify the proteolytic activity that was degrading hGH protein in vivo, various protease inhibitors were tested. The addition of chymostatin, a specific inhibitor of chymotrypsin- and subtilisin-like proteases, to IF extracts, completely inhibited further degradation of the intact hGH protein. In addition, PMSF, a general serine protease inhibitor, and potato PI-I, a specific inhibitor of chymotrypsin-like serine protease activity also inhibited further degradation of the recombinant protein.

Although the invention has been described with reference to the presently preferred embodiments, it should be understood that various modifications can be made without departing from the spirit of the invention.

What is claimed is:

1. A method of changing the phenotype or biochemistry of a plant, comprising:
(a) expressing transiently a nucleic acid sequence from a donor plant in a positive sense orientation in a host plant, wherein said nucleic acid from said donor plant has not been identified;
(b) determining one or more phenotypic or biochemical changes in said host plant.

2. A method of changing the phenotype or biochemistry of a plant, comprising:
(a) expressing transiently a nucleic acid sequence from a donor plant in a positive sense orientation in a host plant, wherein said donor plant and said host plant belong to a different family, order, class, subdivision, or division;
(b) determining one or more phenotypic or biochemical changes in said host plant.

3. A method of determining a change in phenotype or biochemistry in a plant due to a transient expression of a nucleic acid in a positive sense orientation, comprising:
(a) expressing transiently a nucleic acid sequence from a donor plant in a positive sense orientation in a host plant, wherein said nucleic acid from said donor plant has not been identified;
(b) determining one or more biochemical or phenotypic changes in said host plant; and
(c) correlating said one or more biochemical or phenotypic changes to a host plant that is uninfected.

4. A method of determining a change in phenotype or biochemistry in a plant due to a transient expression of a nucleic acid in a positive sense orientation, comprising:
(a) expressing transiently a nucleic acid sequence from a donor plant in a positive sense orientation in a host plant, wherein said donor plant and said host plant belong to a different family, order, class, subdivision, or division;
(b) determining one or more biochemical or phenotypic changes in said host plant; and
(c) correlating said one or more biochemical or phenotypic changes to a host plant that is uninfected.

5. A method of determining the presence of a trait in a plant, comprising:
(a) expressing transiently a nucleic acid sequence from a donor plant in a positive sense orientation in a host plant, wherein said nucleic acid from said donor plant has not been identified;
(b) determining one or more biochemical or phenotypic changes in said host plant;
(c) correlating said one or more biochemical or phenotypic changes to a host plant that is uninfected; and
(d) identifying a trait present in said uninfected host plant.

6. A method of determining the presence of a trait in a plant, comprising:
(a) expressing transiently a nucleic acid sequence from a donor plant in a positive sense orientation in a host plant, wherein said donor plant and said host plant belong to a different family, order, class, subdivision, or division;
(b) determining one or more biochemical or phenotypic changes in said host plant;
(c) correlating said one or more biochemical or phenotypic changes to a host plant that is uninfected; and
(d) identifying a trait present in said uninfected host plant.

7. A method of determining the presence of a trait in a plant, comprising:
(a) expressing transiently a nucleic acid sequence from a donor plant in a positive sense orientation in a host plant, wherein said nucleic acid from said donor plant has not been identified;
(b) determining one or more biochemical or, phenotypic changes in said host plant;
(c) correlating said one or more biochemical or phenotypic changes to a host plant that is uninfected; and
(d) identifying a trait present in said infected host plant.

8. A method of determining the presence of a trait in a plant, comprising:
(a) expressing transiently a nucleic acid sequence from a donor plant in a positive sense orientation in a host plant, wherein said donor plant and said host plant belong to a different family, order, class, subdivision, or division;
(b) determining one or more biochemical or, phenotypic changes in said host plant;
(c) correlating said one or more biochemical or phenotypic changes to a host plant that is uninfected; and
(d) identifying a trait present in said infected host plant.

9. The method according to any one of claims 3-8, further comprises the step of correlating said one or more biochemical or phenotypic changes to a host plant that is infected with a viral vector that contains a known nucleic acid sequence but in a positive sense orientation, wherein said known nucleic acid sequence has similar size but is different in sequence from said nucleic acid sequence in (a).

10. The method according to any one of claims 1-4, wherein said donor plant is selected from the group consisting of food crops, seed crops, oil crops, ornamental crops and forestry crops.

11. The method according to any one of claims 1-4, wherein said host plant is selected from the group consisting of food crops, seed crops, oil crops, ornamental crops and forestry.

12. The method according to claim 11, wherein said host plant is Nicotiana.

13. The method according to claim 12, wherein said host plant is Nicotiana benthamina or Nicotiana cleavlandii.

14. The method according to any one of claims 1-4, wherein said nucleic acid sequence is derived from a library of cDNAs, genomic DNAs, or a pool of RNAs, which represents all or part of the donor plant genome.

15. The method according to claim 14, further comprising the step of cloning said nucleic acid sequence into a plant viral vector.

16. The method according to claim 14, wherein the plant viral vector genome is capped or uncapped.

17. The method according to claim 14, further comprising the step of infecting said host plant with a recombinant viral nucleic acid comprising said nucleic acid sequence.

18. The method according to claim 17, wherein said recombinant viral nucleic acid further comprises a native plant viral subgenomic promoter and a plant viral coat protein coding sequence.

19. The method according to claim 18, wherein said recombinant viral nucleic acid further comprises a non-native plant viral subgenomic promoter, said native plant viral subgenomic promoter initiates transcription of said plant viral coat protein sequence and said non-native plant viral subgenomic promoter initiates transcription of said nucleic acid sequence.

20. The method according to claim 17, wherein a plus sense RNA is produced in the cytoplasm of said host plant, and said plus sense RNA results in overexpression of a protein in said host plant.

21. The method according to claim 17, wherein a plus sense RNA is produced in the cytoplasm of said host plant, and said plus sense RNA results in an enhanced or reduced expression of an endogenous gene in said host plant.

22. The method according to claim 21, wherein said nucleic acid sequence encodes a GTP binding protein.

23. The method according to claim 21, wherein said plus sense RNA results in a reduced expression of an endogenous gene in said host plant.

24. The method according to claim 23, wherein said nucleic acid sequence does not contain a start codon.

25. The method according to claim 23, wherein said nucleic acid encodes an untranslated region.

26. The method according to claim 17, wherein said recombinant viral nucleic acids are derived from a plant virus.

27. The method according to claim 26, wherein said plant virus is selected from the group consisting of a polyvirus, a tobamovirus, a bromovirus, and a geminivirus.

28. The method according to claim 17, wherein said polyvirus is a rice necrosis virus.

29. The method according to any one of claims 1-4, wherein said phenotypic changes are changes in growth rates, morphology, or color.

30. A method of determining the presence of a trait in a plant, comprising:
(a) expressing transiently a nucleic acid sequence of a donor plant in a positive sense orientation in a host plant,
(b) determining phenotypic or biochemical changes in said host plant, and
(c) correlating said expression with said phenotypic or biochemical changes, wherein said nucleic acid sequence comprising a GTP binding protein open reading frame having a positive sense orientation.

31. The method according to claim 30, wherein said GTP binding protein is selected from the group consisting of rab family, and ADP-ribosylation factor family.

32. A method for identifying a nucleic acid sequence in a donor plant having the same function as that in a host plant that belongs to a different genus, family, order, class, subdivision, or division from said donor plant, said method comprising the steps of:
(a) preparing a library of cDNAs, genomic DNAs, or a pool of RNAs of said donor plant,
(b) constructing recombinant viral nucleic acids comprising a nucleic acid insert derived from said library,
(c) infecting each said host plant with one of said recombinant viral nucleic acids, and expressing said nucleic acid in a positive sense orientation in said host plant,
(d) growing said infected host plant,
(e) determining one or more changes in said host plant,
(f) identifying said recombinant viral nucleic acid that results in changes in said host plant, and
(g) determining the sequence of said nucleic acid insert in said recombinant viral nucleic acid, and
(h) determining the sequence of an entire open reading frame of said donor from which said nucleic acid insert is derived.

33. A method for identifying a nucleic acid sequence in a host plant having the same function as that in a donor plant that belongs to a different genus, family, order, class, subdivision, division, or subkingdom from said donor plant, said method comprising the steps of:
(a) preparing a library of cDNAs, genomic DNAs, or a pool of RNAs of said donor plant,
(b) constructing recombinant viral nucleic acids comprising a nucleic acid insert derived from said library,
(c) infecting each said host plant with one of said recombinant viral nucleic acids, and expressing said nucleic acid in a positive sense orientation in said host plant,
(d) growing said infected host plant,
(e) determining one or more changes in said host plant,
(f) identifying said recombinant viral nucleic acid that results in changes in said host plant, and
(g) determining the sequence of said nucleic acid insert in said recombinant viral nucleic acid, and
(h) determining the sequence of an entire open reading frame of a gene in said host plant, the expression of which gene is affected by said insert.

34. A method for detecting the presence of a nucleic acid sequence that has homology in a donor plant and in a host plant, wherein said donor plant and said host plant belong to a different genus, family, order, class, subdivision, or division, said method comprising the steps of:
(a) preparing a library of cDNAs, genomic DNAs, or a pool of RNAs of said donor plant,
(b) constructing recombinant viral nucleic acids comprising a nucleic acid insert derived from said library,
(c) infecting each said host plant with one of said recombinant viral nucleic acids, and expressing said nucleic acid in a positive sense orientation in said host plant,
(d) growing said infected host plant, and
(e) detecting one or more changes in said host plant.

35. A method for determining a nucleic acid sequence that has homology in a donor plant and in a host plant, wherein said donor plant and said host plant belong to a different genus, family, order, class, subdivision, or division, said method comprising the steps of:
(a) preparing a library of cDNAs, genomic DNAs, or a pool of RNAs of said donor plant,
(b) constructing recombinant viral nucleic acids comprising a nucleic acid insert derived from said library,
(c) infecting each said host plant with one of said recombinant viral nucleic acids, and expressing said nucleic acid in a positive sense orientation in said host plant,
(d) growing said infected host plant,
(e) detecting one or more changes in said host plant,
(f) identifying said recombinant viral nucleic acid that results in changes in said host plant, and
(g) determining the sequence of said nucleic acid insert in said recombinant viral nucleic acid, and
(h) determining the sequence of an entire open reading frame of said donor from which said nucleic acid insert is derived.

36. The method according to any one of claims 32-35, wherein said nucleic acid sequence comprising a GTP binding protein open reading frame having a positive sense orientation.

37. A method of increasing yield of a grain crop, said method comprising expressing transiently a nucleic acid sequence of a donor plant in a positive sense orientation in said grain crop, wherein said expressing results in stunted growth and increased seed production of said grain crop.

38. The method according to claim 37, further comprising the step of cloning said nucleic acid sequence into a plant viral vector.

39. The method according to claim 38, further comprising infecting said grain crop with a recombinant viral nucleic acid comprising said nucleic acid sequence.

40. The method according to claim 39, wherein said nucleic acid comprises a GTP binding protein open reading frame having a positive sense orientation.

41. The method according to claim 37, wherein said grain crop is rice.

42. The method according to claim 37, wherein said plant viral vector is derived from a virus selected from the group consisting of a Brome Mosaic virus, a Rice Necrosis virus, and a geminivirus.

43. A method of compiling a plant positive sense functional gene profile comprising:
(a) preparing a library of DNA or RNA sequences from a donor plant, and constructing recombinant viral nucleic acids comprising an unidentified nucleic acid insert obtained from said library in a positive sense orientation;
(b) infecting a plant host with one or more said recombinant viral nucleic acids;
(c) transiently expressing said unidentified nucleic acid in said plant host;
(d) determining one or more phenotypic or biochemical changes in said plant host;
(e) identifying a trait associated with said one or more phenotypic or biochemical changes;
(f) identifying said recombinant viral nucleic acid that results in said one or more changes in plant host;
(g) repeating steps b)-f) until at least one nucleic acid associated with said trait is identified, whereby a positive sense functional gene profile of said plant host or said donor plant is compiled.

44. A method of compiling a plant positive sense functional gene profile comprising:
a) preparing a library of DNA or RNA sequences from a donor plant, and constructing recombinant viral nucleic acids comprising an unidentified nucleic acid insert obtained from said library;
b) infecting a plant host with one or more said recombinant viral nucleic acids;
c) transiently expressing said recombinant nucleic acid in the plant host;
d) determining one or more phenotypic or biochemical changes in the plant host;
e) identifying a trait associated with said one or more phenotypic or biochemical changes;
f) identifying the recombinant viral nucleic acid that results in said one or more changes in the plant host;
g) determining and selecting the sequence of said nucleic acid insert in said recombinant viral nucleic acid that is in a positive sense orientation; and
h) repeating steps b)-g) until at least one nucleic acid sequence associated
with said trait is identified, whereby a positive sense functional gene profile of the plant host or the donor plant is compiled.

45. The method according to claim 43 or 44, further comprising a step of identifying a donor plant gene associated with said trait.

46. The method according to claim 43 or 44, further comprising a step of identifying a host plant gene associated with said trait.

47. The method according to claim 43, wherein said plant host is Nicotiana.

48. The method according to claim 44, wherein said plant host is Nicotiana benthamina or Nicotiana cleavlandii.

49. The method according to claim 43, wherein a positive sense RNA is produced in the cytoplasm of said plant host, and said positive sense RNAs results in a reduced or enhanced expression of an endogenous gene in said host plant.

50. The method according to claim 43, wherein a positive sense RNA is produced in the cytoplasm of said host plant, and said positive sense RNA results in overexpression of a protein in said host plant.

51. The method according to claim 43, wherein said recombinant viral nucleic acid further comprises a native plant viral subgenomic promoter and a plant viral coat protein coding sequence.

52. The method according to claim 51, wherein said recombinant viral nucleic acid further comprises a non-native plant viral subgenomic promoter, said native plant viral subgenomic promoter initiates transcription of said plant viral coat protein sequence and said non-native plant viral subgenomic promoter initiates transcription of said nucleic acid sequence.

53. The method according to claim 43 or 44, wherein said recombinant viral nucleic acids are obtained from a plant virus.

54. The method according to claim 50, wherein said plant virus is a single-stranded plus sense RNA virus.

55. The method according to claim 51, wherein said plant virus is selected from the group consisting of a polyvirus, a tobamovirus, and a bromovirus.

56. The method according to claim 55, wherein said tobamovirus is a tobacco mosaic virus.

57. The method according to claim 55, wherein said polyvirus is a rice necrosis virus.


SEQUENCE LISTING (48 sequences)

SEQ ID NO: 1 - DNA, 26 bp, Artificial Sequence
  Artificially generated primer
ctcgcaaagtttcgaaccaaatcctc

SEQ ID NO: 2 - DNA, 35 bp, Artificial Sequence
  Artificially generated primer
cggggtacctgggccccaaccgggggttccggggg

SEQ ID NO: 3 - DNA, 41 bp, Artificial Sequence
  Artificially generated primer
tcctcgagcctaggctcgcaaagtttcgaaccaaatcctca

SEQ ID NO: 4 - DNA, 35 bp, Artificial Sequence; identical to SEQ ID NO: 2
  Artificially generated primer

SEQ ID NO: 5 - DNA, 24 bp, Artificial Sequence
  Artificially generated primer
tatgtatggtgcagaagaacagat

SEQ ID NO: 6 - DNA, 25 bp, Artificial Sequence
  Artificially generated  primer
agtcgactcttcctcttctggcatc

SEQ ID NO: 7 - DNA, 30 bp, Artificial Sequence
  Artificially generated primer
tgctcgagtgtgttcttcagttttctgtca

SEQ ID NO: 8 - DNA, 30 bp, Artificial Sequence
  Artificially generated primer
aactcgagcgctttgatttctccgaagctt

SEQ ID NO: 9 - DNA, 114 bp, Tomato mosaic virus
  CDS (28)...(114)
gttttaaatacgctcgaggttttaaatatgtctgttgccttgttatgggttgtttctccttgtgacgtctcaaatgggacaagtttcatggaatcagtccgggagggaaaccgt

SEQ ID NO: 10 - protein, 29 aa, Tomato mosaic virus
MSVALLWVVSPCDVSNGTSFMESVREGNR

SEQ ID NO: 11 - DNA, 39 bp, Artificial Sequence
  Artificially generated primer
gcctcgagtgcagcatggaaacccttctaaagcttttcc

SEQ ID NO: 12 - DNA, 36 bp, Artificial Sequence
  Artificially generated primer
tccctaggtcaaaggctctctattgctagattgccc

SEQ ID NO: 13 - DNA, 42 bp, Artificial Sequence
  Artificially generated primer
gcctcgagtgcagcatgatcagattcttagtcctctctttga

SEQ ID NO: 14 - DNA, 36 bp, Artificial Sequence
  Artificially generated primer
tccctaggctaaatagcataacttccacatcaaagc

SEQ ID NO: 15 - DNA, 31 bp, Artificial Sequence
  Artificially generated primer
tactcgaggttcataagaccgcggtaggcgg

SEQ ID NO: 16 - DNA, 36 bp, Artificial Sequence
  Artificially generated primer
cggggtacctgggcccctacccggggtttagggagg

SEQ ID NO: 17 - DNA, 1543 bp, Ribgrass mosaic virus (RMV)
ctcgaggttcataagaccgcggtaggcggagcgtttgtttactgtagtataattaaatatttgtcagataaaaggttgtttaaagatttgttttttgtttgactgagtcgataatgtcttacgagcctaaagttagtgacttccttgctcttacgaaaaaggaggaaattttacccaaggctttgacgagattaaagactgtctctattagtactaaggatgttatatctgttaaggagtctgagtccctgtgtgatattgatttgttagtgaatgtgccattagataagtataggtatgtgggtgttttgggtgttgttttcaccggtgaatggctggtaccggatttcgttaaaggtggggtaacagtgagcgtgattgacaaacggcttgaaaattccagagagtgcataattggtacgtaccgagctgctgtaaaggacagaaggttccagttcaagctggttccaaattacttcgtatccattgcggatgccaagcgaaaaccgtggcaggttcatgtgcgaattcaaaatctgaagatcgaagctggatggcaacctctagctctagaggtggtttctgttgccatggttactaataacgtggttgttaaaggtttgagggaaaaggtcatcgcagtgaatgatccgaacgtcgaaggtttcgaaggtgtggttgacgatttcgtcgattcggttgctgcattcaaggcgattgacagtttccgaaagaaaaagaaaaagattggaggaagggatgtaaataataataagtatagatatagaccggagagatacgccggtcctgattcgttacaatataaagaagaaaatggtttacaacatcacgagctcgaatcagtaccagtatttcgcagcgatgtgggcagagcccacagcgatgcttaaccagtgcgtgtctgcgttgtcgcaatcgtatcaaactcaggcggcaagagatactgttagacagcagttctctaaccttctgagtgcgattgtgacaccgaaccagcggtttccagaaacaggataccgggtgtatattaattcagcagttctaaaaccgttgtacgagtctctcatgaagtcctttgatactagaaataggatcattgaaactgaagaagagtcgcgtccatcggcttccgaagtatctaatgcaacacaacgtgttgatgatgcgaccgtggccatcaggagtcaaattcagcttttgctgaacgagctctccaacggacatggtctgatgaacagggcagagttcgaggttttattaccttgggctactgcgccagctacataggcgtggtgcacacgatagtgcatagtgtttttctctccacttaaatcgaagagatatacttacggtgtaattccgcaagggtggcgtaaaccaaattacgcaatgttttaggttccatttaaatcgaaacctgttatttcctggatcacctgttaacgtacgcgtggcgtatattacagtgggaataactaaaagtgagaggttcgaatcctccctaaccccgggtaggggccca

SEQ ID NO: 18 - DNA, 55 bp, Artificial Sequence
  Artificially generated primer
gatggcgccttaatacgactcactatagttttatttttgttgcaacaacaacaac

SEQ ID NO: 19 - DNA, 30 bp, Artificial Sequence
  Artificially generated primer
cttgtgcccttcatgacgagctatatcacg

SEQ ID NO: 20 - DNA, 496 bp, Ribgrass mosaic virus (RMV)
ccttaatacgactcactatagttttatttttgttgcaacaacaacaacaaattacaataacaacaaaacaaatacaaacaacaacaacatggcacaatttcaacaaacagtaaacatgcaaacattgcaggctgccgcagggcgcaacagcctggtgaatgatttagcctcacgacgtgtttatgacaatgctgtcgaggagctaaatgcacgctcgagacgccctaaggttcattactccaaatcagtgtctacggaacagacgctgttagcttcaaacgcttatccggagtttgagatttcctttactcatacccaacatgccgtacactcccttgcgggtggcctaaggactcttgagttagagtatctcatgatgcaagttccgttcggttctctgacgtacgacatcggtggtaactttgcagcgcaccttttcaaaggacgcgactacgttcactgctgtatgccaaacttggatgtacgtgatatagct

SEQ ID NO: 21 - DNA, 55 bp, Artificial Sequence; identical to SEQ ID NO: 18
  Artificially generated primer

SEQ ID NO: 22 - DNA, 37 bp, Artificial Sequence
  Artificially generated primer
atcgtttaaactgggcccctacccggggttagggagg

SEQ ID NO: 23 - DNA, 497 bp, Ribgrass Mosaic virus (RMV)
ccttaatacgactcactatagttttatttttgttgcaacaacaacaacaaattacaataacaacaaaacaaatacaaacaacaacaacatggcacaatttcaacaaacagtaaacatgcaaacattccaggctgccgcagggcgcaacagcctggtgaatgatttagcctcacgacgtgtttatgacaatgctgtcgaggagctaaatgcacgctcgagacgccctaaggttcattactccaaatcagtgtctacggaacagacgctgttagcttcaaacgcttatccggagtttgagatttcctttactcatacccaaacatgccgtacactcccttgcgggtggcctaaggactcttgagttagagtatctcatgatgcaagttccgttcggttctctgacgtacgacatcggtggtaactttgcagcgcaccttttcaaaggacgcgactacgttcactgctgtatgccaaacttggatgtacgtgatatagct

SEQ ID NO: 24 - DNA, 55 bp, Artificial Sequence; identical to SEQ ID NO: 18
  Artificially generated primer

SEQ ID NO: 25 - DNA, 37 bp, Artificial Sequence; identical to SEQ ID NO: 22
  Artificially generated primer

SEQ ID NO: 26 - DNA, 14 bp, Artificial Sequence
  Artificially generated primer
tcgagcggccgcat

SEQ ID NO: 27 - DNA, 8 bp, Artificial Sequence
  Artificially generated primer
gcggccgc

SEQ ID NO: 28 - protein, 19 aa, P. yoelii
SYVPSAEQILEFVKQISSQ

SEQ ID NO: 29 - DNA, 956 bp, Arabidopsis thaliana
  CDS (15)...(675)
cttcactttcgccgatggctctacctaaccagcaaaccgtggattaccctagcttcaagctcgttatcgttggcgatggaggcacagggaagaccacatttgtaaagagacatcttactggagagtttgagaagaagtatgaacccactattggtgttgaggttcatcctcttgatttcttcactaactgtggcaagatccgtttctactgttgggatactgctggccaagagaaatttggtggtcttagggatggttactacatccatggacaatgtgctatcatcatgtttgatgtcacagcacgactgacatacaagaatgttccaacatggcaccgtgatctttgcagggtttgtgaaaacatcccaattgttctttgtgggaataaagttgatgtgaagaacaggcaagtcaaggccaacatcccaattgttctttgtgggaataaagttgatgtgaagaacagggtttgtgaaaacatcccaattgttctttgtgggaataaagttgatgtgaagaacaggcaagtcaaggccaagcaggtaacattccacaggaagaagaacctccagtattacgagatatctgccaagagcaactacaacttcgagaagccattcttgtaccttgctagaaagctcgccggggacgctaatcttcactttgtggaatcacctgcccttgctcccccggaagttcaaatcgacttggctgctcagcagcagcatgaggcggagcttgcagcagcagcaagtcagccacttcctgatgacgatgatgacaccttcgagtagagaaagagagatgtgatctgtcactgattacccgttagggcttgtctgaacttttttttgttcatggtgctatttttatgtgtccgtactttgaaatgaatcgatgacattagtaattttcatttttaagtttttaactgtcgctatgaaagtgaaaac

SEQ ID NO: 30 - protein, 220 aa, Arabidopsis thaliana
MALPNQQTVDYPSFKLVIVGDGGTGKTTFVKRHLTGEFEKKYEPTIGVEVHPLDFFTNCGKIRFYCWDTAGQEKFGGLRDGYYIHGQCAIIMFDVTARLTYKNVPTWHRDLCRVCENIPIVLCGNKVDVKNRQVKANIPIVLCGNKVDVKNRVCENIPIVLCGNKVDVKNRQVKAKQVTFHRKKNLQYYEISAKSNYNFEKPFLYLARKLAGDANLHFVE

SEQ ID NO: 31 - DNA, 798 bp, Arabidopsis thaliana
cttcactttcgccgatggctctacctaaccagcaaaccgtggattaccctagcttcaagctcgttatcgttggcgatggaggcacagggaagaccacatttgtaaagagacatcttactggagagtttgagaagaagtatgaacccactattggtgttgaggttcatcctcctgatttcttcactaactgtggcaagatccgtttctactgttgggatactgctggccaagagaaatttgtcactaactgtggcaagatccgtttctactgttgggatactgctggccaagagaaatttggtggtcttagggatggttactacatccatggacaatgtgctatcatcatgtttgatgtcacagcacgactgacatacaggaatgttccaacatggcaccgtgatctttgcagggtttgtgaaaacatcccaattgttctttgtgggaataaagttgatgtgaagaacaggcaagtcaaggccaagcaggtaacattccacaggaaggaggaactccagtattacgagatatctgccaagagcaactacaacttcgagaagccattcttgtaccttgctagaaagctcgccggggacgctaatcttcactttgtggaatcacctgcccttgctcccccggaagttcaaatcgacttggctgctcagcagcagcatgaggcggagcttgcagcagcagcaagtcagccacttcctgatgacgatgatgacaccttcgagtagagaaagagagatgtgatctgtcactgattacccgttagggcttgtctgaacttttttt

SEQ ID NO: 32 - DNA, 667 bp, N. tabacum
atggctctaccaaaccaacaaactgtagattatccaagcttcaagcttgtaatcgtgggcgatggaggaactgggaaaacaacttttgtcaagaggcatcttactggtgaatttgagaagaaatatgaacccactattggtgtggaggttcatccattagacttcttcacaaattgtgggaaaattcgcttttattgctgggatactgctggacaagagaagtttggaggtcttcgggatggttactacattcatgggcaatgcgcaattatcatgtttgatgttacagcccgtctgacctacaagaatgttcctacgtggcatcgagatctctgcagggtttgtgaaaacatccccattgttctttgtggaaacaaagttgatgtcaagaacaggcaggttaaggcaaagcaagttaccttccacaggaagaaaaatttgcaatactatgagatctcagcaaagagtaactacaactttgagaagccttttctgtaccttgccagaaagcttgctggggatgctaatcttcactttgtggaatcacctgcacttgctccccctgaagtacaaattgatttagctgcacagcaactgcatgaacaagagcttttgcaagccgctgcgcacgcacttccagatgacgatgatgaagcttttgaataga

SEQ ID NO: 33 - protein, 135 aa, Arabidopsis thaliana
MALPNQQTVDYPSFKLVIVGDGGTGKTTFVKRHLTGEFEKKYEPTIGVEVHPLDFFTNCGKIRFYCWDTAGQEKFGGLRDGYYIHGQCAIIMFDVTSTTDIQECSNMAPSLQGLKHSQLFFVGIKLMKNRQVKAQ

SEQ ID NO: 34 - protein, 137 aa, Tobacco RAN-B1
MALPNQQTVDYPSFKLVIVGDGGTGKTTFVKRHLTGEFEKKYEPTIGVEVHPLDFFTNCGKIRFYCWDTAGQEKFGGLRDGYYIHGQCAIIMFDVTARLTYKNVPTWHRDLCRVCENIPIVLCGNKVDVKNRQVKAK

SEQ ID NO: 35 - DNA, 278 bp, Arabidopsis thaliana
gctactatggttgcctctccggctcaggccactatggtcgctccattcaacggacttaagtcctccgctgccttcccagccacccgcaaggctaacaacgacattacttccatcacaagcaacggcggaagagttaactgcatgcaggtgtggcctccgattggaaagaagaagtttgagactctctcttaccttcctgaccttaccgattccgaattggctaaggaagttgactaccttatccgcaacaagtggattccttgtgttgaattcgaagt

SEQ ID NO: 36 - protein, 93 aa, Arabidopsis thaliana
ATMVASPAQATMVAPFNGLKSSAAFPATRKANNDITSITSNGGRVNCMQVWPPIGKKKFETLSYLPDLTDSELAKEVDYLIRNKWIPCVEFEV

SEQ ID NO: 37 - DNA, 168 bp, Arabidopsis thaliana
acttgatctgtttcatactaaaaccaaaactcatgtttgttcactccaaacacaaacacagcagtaatcaaaaatcgtcttataacaaaaaggaaatgcaacaaaacagaagaaacaactaagtagtaggcaagattcttcttcactcgtcttcttggctacggagcc

SEQ ID NO: 38 - DNA, 379 bp, Arabidopsis thaliana
gaaacgacgtcggctagttattgggcatggcctgaccagcagcaacaacatcacaatcatcatcagttcaattgatcatattgtctaagaacaacatcatactcatcttgatatcatattatcatcaaaagaaaattccgtagatttttttaataagtattttcaaattatttggcacgtttaaaattaattaaattgggttattatgtttacttgatctgtttcatactaaaaccaaaactcatgtttgttcactccaaacacaaacacagcagtaatcaaaaatcgtcttataacaaaaaggaaatgcaacaaaacagaagaaacaactaagtagtaggcaagattcttcttcactcgtcttcttggctacggagcc

SEQ ID NO: 39 - protein, 24 aa, Arabidopsis thaliana
ETTSASYWAWPDQQQQHHNHHQFN

SEQ ID NO: 40 - DNA, 11 bp, Tobacco mosaic virus
tatagtatttt

SEQ ID NO: 41 - DNA, 11 bp, Tobacco mosaic virus
tataggtattt

SEQ ID NO: 42 - DNA, 11 bp, Artificial Sequence
  Artificially generated promoter n=(a or g or c or t)
tatagntattt

SEQ ID NO: 43 - DNA, 12 bp, Artificial Sequence
  Artificially generated promoter
tataggtatttt

SEQ ID NO: 44 - DNA, 14 bp, Artificial Sequence
  Artificially generated promoter
tatagtcgtatttt

SEQ ID NO: 45 - DNA, 15 bp, Artificial Sequence
  Artificially generated promoter n=(a or g or c or t)
tatagtngtngtatt

SEQ ID NO: 46 - DNA, 21 bp, Artificial Sequence
  Artifically generated promoter n=(a or g or c or t)
tatagtngtngtngtngtatt

SEQ ID NO: 47 - DNA, 16 bp, Artificial Sequence
  Artifically generated promoter
tatagtatttgtattt

SEQ ID NO: 48 - DNA, 12 bp, Artificial Sequence
  Artificially generated promoter
tatagtcgtatt